(19) United States
(12) Patent Application Publication
(10) Pub. No.: US 2024/0024457 A1
(43) Pub. Date: Jan. 25, 2024

(54) DIFFERENTIAL COATING OF MICROPROJECTIONS AND MICRONEEDLES ON ARRAYS

(71) Applicant: Vaxxas Pty Limited, Sydney (AU)

(72) Inventors: Michael Carl JUNGER, Brookfield (AU); Christopher FLAIM, Chapel Hill (AU); Paul FAHEY, Milton (AU); Charlotte SWEENEY, Milton (AU); Senhil MURUGAPPAN, Milton (AU); Paul KELLY, Milton (AU); Angus FOSTER, Milton (AU)

(21) Appl. No.: 18/356,837

(22) Filed: Jul. 21, 2023

Related U.S. Application Data
(63) Continuation of application No. 16/638,072, filed on Feb. 10, 2020, now abandoned.
(60) Provisional application No. 62/605,401, filed on Aug. 10, 2017.

(51) Int. Cl. A61K 39/145; A61K 9/00; A61K 39/13; C12N 7/00

(52) U.S. Cl. CPC A61K 39/145; A61K 9/0021; A61K 39/13; C12N 7/00; A61B 17/205

(57) ABSTRACT

The present invention relates to devices and methods for coating microprojection or microneedle arrays including arrays that contain vaccine formulations, more specifically to multivalent vaccine formulations where components of the multivalent vaccine might be incompatible. The present invention further relates to stable vaccine formulations for administration via a microprojection array in which the microprojections are densely packed and in which the vaccine formulations are sprayed on to the microprojections such that the formulations dry quickly

### BACKGROUND OF THE INVENTION

The present invention relates to devices and methods for coating microprojection or microneedle arrays including arrays that contain vaccine formulations, more specifically to multivalent vaccine formulations where components of the multivalent vaccine might be incompatible. The present invention further relates to stable vaccine formulations for administration via a microprojection array in which the microprojections are densely packed and in which the vaccine formulations are rapidly sprayed or layered on to the microprojections in relatively small amounts such that the formulations dry rapidly.

### DESCRIPTION OF THE PRIOR ART

In recent years, attempts have been made to devise new methods of delivering drugs and other bioactive materials, for vaccination and other purposes, which provide alternatives that are more convenient and/or enhanced in performance to the customary routes of administration such as intramuscular and intradermal injection. Limitations of intradermal injection include: cross-contamination through needle-stick injuries in health workers; injection phobia from a needle and syringe; and most importantly, as a result of its comparatively large scale and method of administration, the needle and syringe cannot target key cells in the outer skin layers. This is a serious limitation to many existing and emerging strategies for the prevention, treatment and monitoring of a range of untreatable diseases. There is also a need to reduce the amount of material delivered due to toxicity of the material or due to the need to conserve the material because it is difficult and/or expensive to produce.

In an effort to solve some of the issues referenced above microprojection arrays or microneedle arrays have been utilized to deliver various materials through the skin. For example, WO 2005/072630 describes devices for delivering bioactive materials and other stimuli to living cells. The devices comprise a plurality of projections which can penetrate the skin so as to deliver a bioactive material or stimulus to a predetermined site. The projections can be solid and the delivery end of the projection is designed such that it can be inserted into targeted cells or specific sites on the skin.

One of the challenges of using devices that contain microneedles and/or microprojections is the need to coat the projections. Various coating techniques such as dipping the array into a coating solution or spraying the coating onto the projections have been described. For example, Gill and Prausnitz, J. Controlled Release (2007), 117: 227-237 describe coating microprojections by dipping the microprojections into a coating solution reservoir through dip holes that are spaced in accordance with the microprojection array. Cormier et al., J. Controlled Release (2004), 97: 503-511 describe coating microneedle arrays by partial immersion in an aqueous solution containing active compounds and polysorbate. WO 2009/079712 describes methods for coating microprojection arrays by spray coating the microprojections and drying the sprayed solution with gas.

Inkjet printing has been use to deposit pharmaceutical compositions on a variety of devices and media. For example Wu et al., (1996) J. Control. Release 40: 77-87 described the use of inkjets to creating devices containing model drugs; Radulescu et al. (2003) Proc. Winter Symposium and 11th International Symposium on Recent Advance ins Drug Delivery Systems described the preparation of small diameter poly(lactic-co-glycolic acid) nanoparticles containing paclitaxel using a piezoelectric inkjet printer; Melendez et al. (2008) J. Pharm. Sci. 97: 2619-2636 utilized inkjet printers to produce solid dosage forms of prednisolone; Desai et al. (2010) Mater. Sci. Eng. B 168: 127-131 used a piezoelectric inkjet printer to deposit sodium alginate aqueous solutions containing rhodamine R6G dye onto calcium chloride surfaces; Sandler et al. (2011) J. Pharm. Sci. 100: 3386-3395 used inkjet printing to deposit various pharmaceutical compounds on porous paper substrates; Scoutaris et al. (2012) J. Mater. Sci. Mater. Med. 23: 385-391 described the use of inkjet printing to create a dot array containing two pharmacological agents and two polymers. Inkjet printing has also been used to deposit various pharmaceutical compositions on stents (Tarcha, et al. (2007) Ann. Biomed. Eng. 35: 1791-1799). Recently, piezoelectric inkjet printers have been used to coat microneedles. Boehm et al. (2014) Materials Today 17(5): 247-252 has described the use of inkjet printers to coat microneedles prepared from a biodegradable acid anhydride compolymer which contains alternating methyl vinyl ether and maleic anhydride groups with miconazole.

Rapid spray coating of microprojection/microneedle drug delivery and vaccine platforms allow allocation of the coating to the delivery platform minimizing the inefficiencies associated with spray coating or dip coating that may overcoat or undercoat the microprojections. Moreover, dip coating or spray coating is less accurate than ink jet coating. Many vaccines are comprised of multiple valencies that may be for protection against a single pathogen such as a thirteen valent vaccine against pneumococcal infections or multiple pathogens (multiple actives) such as MMR vaccine against measles mumps and rubella. Such vaccines containing more than one active may have incompatibilities among the various actives or among the various excipients or solvents used to deliver the vaccine or to make the vaccine more efficacious. Moreover, designing a stable vaccine with multiple valencies that may be distributed on a surface such as a microneedle or microprojection and dried poses challenges. In addition each component of the multivalent vaccine composition affects the viscosity, drop formation, dry time, adhesion and stability of the vaccine. Other challenges to delivering a complex vaccine via a microprojection/microneedle array include coating the microneedles/microprojections with enough vaccine to be efficacious when administered, formulating a vaccine such that the drop size is sufficiently small to permit penetration into the skin with each projection of the array. There is also a need to provide microneedle/microprojection arrays that enable coating of the microneedle/microprojection with compositions that have components that are incompatible with each other in solution. In other words, it may be desirable to have microneedle/microprojection arrays that can be coated by a device such that each of the components to be delivered is separately coated on to the microneedle/microprojections.

Although there are clear benefits with combination vaccines, the main challenge in their development is the risk that the efficacy or safety of the combination would be less than that seen with the administration of the vaccines separately. New combinations cannot be less immunogenic, less efficacious, or more reactogenic than the previously licensed uncombined vaccines. Immunological, physical, and/or chemical interactions between the combined components have the potential to alter the immune response to specific components. Finally, and ideally, the many advantages of combination vaccines should not be achieved at the cost of reduced product stability. From a practical standpoint, uncommon transport and storage conditions and could hamper the development of a combination vaccine. Companies have spent years combining vaccine antigens in a single formulation only to discover that one or more of the vaccine components is/are incompatible. If a solution cannot be found, the development of that particular vaccine combination ceases. The present invention provides a delivery mechanism for combination vaccines that negates the need to combine vaccines in the one formulation and therefore completely avoids vaccine component incompatibility. As most existing vaccines can be given concomitantly without interference the present invention of providing devices and methods of delivering multiple vaccines on separate microprojections (or different areas of the same projection) within an array is a significant advancement in the fields of drug delivery and vaccinology.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### SUMMARY OF THE PRESENT INVENTION

The present invention relates to devices and methods for coating microprojection or microneedle arrays with various substances. These substances may be liquid or non-liquid and may be coated onto the microprojection array such that one substance may be coated onto one microprojection and another substance may be coated onto a different microprojection. The methods and devices of the present invention also relate to coating microprojection or microneedle arrays with various substances such that more than one substance is coated onto a given microprojection or microneedle. The multiple substances coating the microprojection or microneedle may completely overcoat one another or partially overcoat one another or the coatings may be such that one substance covers a portion of the microprojection or microneedle and another substance covers another portion of the microprojection or microneedle such that neither substance interacts with the other. The coating of the microprojections or microneedles can include multiple layers such as two layers or more. It is also possible that the microprojections or microneedles are covered with layers that contain the same substance such as in a situation where more substance is needed than can be delivered in a single administration. The present invention also relates to microprojection arrays having a base and a plurality of microprojections where the microprojections are divided into at least different sections or areas where each section or area has a plurality of microprojections and where the microprojections in one of the sections or areas are coated with one substance and where the microprojections in another area or section are coated with a different substance.

The present invention also relates to devices, formulations and methods for coating vaccines onto microprojections of a microprojection array such that the vaccines are more stable than corresponding vaccines is solution. The present invention provides increased stability of vaccine formulations based on antigen activity, such as potency, as measured by various methods including ELISA before and after rapid drying.

The present invention provides increased stability of vaccine formulations based on antigen activity, as measured by various methods including ELISA after drying and storage at various temperatures such 4° C. and 25° C. and elevated temperatures such as 45° C.

The present invention also relates to devices, formulations and methods for increasing the stability of vaccine formulations including but not limited to influenza and inactivated polio vaccine due to the use of excipients which include but are not limited to cyclodextrins, amino acids (such as histidine, arginine, glutamic acid), reducing agents (such as cysteine and glutathione), carbohydrates (such as sucrose and lactose), polymers such as polyethylene glycol or polyvinylpyrrolidone and proteins (such as gelatin) and combinations thereof.

In one broad form an aspect of the present invention seeks to provide a microprojection array comprising a base and a plurality of microprojections, wherein one or more microprojection(s) is coated with two or more substances.

In one embodiment, one or more microprojection(s) is coated with a first substance and a second substance.

In one embodiment, the microprojection is coated such that the first substance overcoats the second substance.

In one embodiment, the microprojection is coated such that the first substance partially overcoats the second substance.

In one embodiment, the microprojection is coated such that the first substance completely overcoats the second substance.

In one embodiment, the microprojection is coated such that the first substance does not overcoat the second substance.

In one embodiment, the microprojection is coated such that the first substance is coated on one side of the microprojection and the second substance on the other side of the microprojection.

In one embodiment, the microprojection is coated such that the first substance is coated on the top of the microprojection and the second substance is coated on the bottom of the microprojection.

In one embodiment, the first substance and the second substance are comprised of one or more vaccine antigens.

In one embodiment, the first substance is an antigen and the second substance is an adjuvant.

In one embodiment, the first substance is an adjuvant and the second substance is an antigen.

In one embodiment, the first substance is in a hydrophobic material and the second substance is a hydrophilic material.

In another broad form an aspect of the present invention seeks to provide a microprojection array comprising a base and a plurality of microprojections, wherein at least a first microprojection is coated with a first substance and at least a second microprojection is coated with a second substance.

In another broad form an aspect of the present invention seeks to provide a microprojection array comprising a base and a plurality of microprojections, wherein a first microprojection is coated with a first substance and a second microprojection is coated with a second substance.

In one embodiment, the first substance is a first multivalent vaccine and the second substance is a second multivalent vaccine.

In one embodiment, the first substance and the second substance are comprised of one or more vaccine antigens.

In one embodiment, the first substance is an antigen and the second substance is an adjuvant.

In one embodiment, the first substance is in a hydrophobic material and the second substance is a hydrophilic material.

In one embodiment, the first substance or the second substance is a contrast enhancing reagent.

In one embodiment, the first substance or the second substance contains a water soluble release substance.

In another broad form an aspect of the present invention seeks to provide a microprojection array comprising a base and a plurality of microprojections, wherein the microprojections are divided into at least a first section and a second section, each section comprising a plurality of microprojections, and wherein the microprojections in the first section are coated with at least a first substance, and wherein the microprojections in the second section are coated with at least a second substance.

In another broad form an aspect of the present invention seeks to provide a microprojection array comprising a base and a plurality of microprojections, wherein the microprojections are divided into at least a first section and a second section, each section comprising a plurality of microprojections, and wherein the microprojections in the first section are coated with a first substance, and wherein the microprojections in the second section are coated with a second substance.

In one embodiment, the first substance is a first multivalent vaccine and the second substance is a second multivalent vaccine.

In one embodiment, the first substance and the second substance are comprised of one or more vaccine antigens.

In one embodiment, the first substance is an antigen and the second substance is an adjuvant.

In one embodiment, the first substance is in a hydrophobic material and the second substance is a hydrophilic material.

In one embodiment, the first substance or the second substance is a contrast enhancing reagent.

In one embodiment, the first substance or the second substance contains a water soluble release substance.

In one embodiment, the first section has at least 100 microprojections.

In one embodiment, the second section has at least 100 microprojections.

In one embodiment, the first section has between 1000 to 10000 microprojections.

In one embodiment, the first section has between 1000 to 10000 microprojections.

In another broad form an aspect of the present invention seeks to provide a method of coating a microprojection array comprising a plurality of microprojections, the method comprising coating the microprojections with a first substance and coating the microprojections with a second substance.

In one embodiment, one or more microprojection(s) is coated with a first substance and a second substance.

In one embodiment, the microprojection is coated such that the first substance overcoats the second substance.

In one embodiment, the microprojection is coated such that the first substance partially overcoats the second substance.

In one embodiment, the microprojection is coated such that the first substance completely overcoats the second substance

In one embodiment, the microprojection is coated such that the first substance does not overcoat the second substance.

In one embodiment, the microprojection is coated such that the first substance is coated on one side of the microprojection and the second substance on the other side of the microprojection.

In one embodiment, the microprojection is coated such that the first substance is coated on the top of the microprojection and the second substance is coated on the bottom of the microprojection.

In one embodiment, the first substance and the second substance are comprised of one or more vaccine antigens.

In one embodiment, the first substance is an antigen and the second substance is an adjuvant.

In one embodiment, the first substance is an adjuvant and the second substance is an antigen.

In another broad form an aspect of the present invention seeks to provide a method of coating a microprojection array comprising two or more sections, each section comprising a plurality of microprojections, the method comprising coating the microprojections in one section with a first substance and coating the microprojections in another section with a second substance.

In one embodiment, the first substance is a first multivalent vaccine and the second substance is a second multivalent vaccine.

In one embodiment, the first substance and the second substance are comprised of one or more vaccine antigens.

In one embodiment, the first substance is an antigen and the second substance is an adjuvant.

In one embodiment, the first substance is in a hydrophobic solvent and the second substance is a hydrophilic solvent.

In one embodiment, the first substance or the second substance is a contrast enhancing reagent.

In one embodiment, the first substance or the second substance contains a water soluble release substance.

In one embodiment, the first section has at least 100 microprojections.

In one embodiment, the second section has at least 100 microprojections.

In one embodiment, the first section has between 1000 to 10000 microprojections.

In one embodiment, the first section has between 1000 to 10000 microprojections.

In another broad form an aspect of the present invention seeks to provide a microprojection array comprising a base and a plurality of microprojections, wherein the number of microprojections is at least 1000 and the density of the microprojections is at least 50 projections/mm², and wherein a first microprojection is adjacent a second microprojection, and wherein the first microprojection is coated with an amount of a first antigen and the second microprojection is coated with an amount of a second antigen.

In one embodiment, the first antigen is hemagglutinin from an H1N1 flu virus and the second antigen is hemagglutinin from B flu virus.

In one embodiment, the first antigen is hemagglutinin from an H3N2 flu virus and the second antigen is hemagglutinin from a B flu virus.

In one embodiment, the microprojection array further comprises a third microprojection adjacent the first and second microprojection wherein the third microprojection is coated with a third antigen.

In one embodiment, the first antigen is hemagglutinin from an H3N2 flu virus and the second antigen is hemagglutinin from a B flu virus and the third antigen is hemagglutinin from an H1N1 flu virus.

In one embodiment, the amount of hemagglutinin from the H3N2 flu virus and the amount of hemagglutinin from B flu virus and the amount of hemagglutinin from H1N1 flu virus is different.

In one embodiment, the amount of hemagglutinin from the H3N2 flu virus is from about 1 μg to about 20 μg and the amount of hemagglutinin from the B flu virus is from about 1 μg to about 20 μg and the amount of hemagglutinin from the H1N1 flu virus is from about 1 μg to about 20 μg.

In another broad form an aspect of the present invention seeks to provide a method of coating materials onto a plurality of microprojections on a microprojection array comprising: applying a first amount of a first material to a first microprojection, wherein the amount is applied such that the first material dries on the projection in less than 3 seconds; and applying a second amount of a second material to a second microprojection, wherein the amount is applied such that the second material dries on the projection in less than 3 seconds, and wherein the second microprojection is directly adjacent the first microprojection, and wherein the second microprojection is about 10 to 200 μm from the first microprojection.

In one embodiment, the first material is a vaccine antigen.

In one embodiment, the second material is a vaccine antigen.

In one embodiment, the first material and the second material are different vaccine antigens.

In one embodiment, the first amount of the first material is different from the second amount of the second material.

In one embodiment, the first material is HA antigen from an A strain of influenza virus.

In one embodiment, the second material is HA antigen from a different A strain of influenza virus as compared to the first material.

In one embodiment, the second material is HA antigen from a B strain of influenza virus.

In one embodiment, the HA antigen from an A strain of influenza virus is stabilized in an excipient selected from the group consisting of arginine, sucrose, sulfobutyl ether β-cyclodextrin, aspartic acid and combinations thereof.

In one embodiment, the amount of excipient is from about 0.5% to about 5.0%.

In one embodiment, the amount of excipient is from about 0.5% to about 2.5%.

In one embodiment, the amount of excipient is from about 0.5% to about 1.5%.

In one embodiment, the excipient is sulfobutyl ether β-cyclodextrin in an amount of from about 0.5% to about 5.0%.

In one embodiment, the first material is a first IPV antigen.

In one embodiment, the second material is a second IPV antigen as compared to the first material.

In one embodiment, the IPV antigen is stabilized in an excipient selected from the group consisting of arginine, sucrose, sulfobutyl ether β-cyclodextrin, Y-cyclodextrin, histidine, glutathione and combinations thereof.

In one embodiment, the amount of excipient is from about 0.5% to about 5.0%.

In one embodiment, the amount of excipient is from about 0.5% to about 2.5%.

In one embodiment, the amount of excipient is from about 0.5% to about 1.5%.

In one embodiment, the excipient is sulfobutyl ether β-cyclodextrin in an amount of from about 0.5% to about 5.0%.

In one embodiment, the excipient is 4.5% SBE β-Cyclodextrin and 15 mM Glutathione.

In one embodiment, the excipient is 2.5% γ-Cyclodextrin and 15 mM Glutathione.

In one embodiment, the IPV is stable for at least 6 months as measured by ELISA.

In another broad form an aspect of the present invention seeks to provide a method of coating materials onto a plurality of microprojections on a microprojection array comprising: applying a vaccine antigen in a formulation to at least one microprojection, wherein the amount is applied such that the antigen dries on the projection in from about 5 ms to 5 seconds, and wherein the antigen the decrease in antigen potency is less than 5% after drying as compared to the antigen in solution.

In one embodiment, the decrease in antigen potency is less than 10% after drying as compared to the antigen in solution.

In one embodiment, the decrease in antigen potency is less than 20% after drying as compared to the antigen in solution.

In one embodiment, the decrease in antigen potency is less than 30% after drying as compared to the antigen in solution.

In one embodiment, the formulation comprises at least one excipient.

In one embodiment, the antigen is an influenza HA antigen.

In one embodiment, the excipient is sulfobutyl ether β-cyclodextrin in an amount of from about 0.5% to about 5.0%.

In one embodiment, the antigen is an IPV antigen.

In one embodiment, the excipient is 4.5% SBE β-Cyclodextrin and 15 mM Glutathione.

In one embodiment, the excipient is 2.5% γ-Cyclodextrin and 15 mM Glutathione.

In one embodiment, the antigen potency is determined by ELISA.

In another broad form an aspect of the present invention seeks to provide a method of coating materials onto a plurality of microprojections on a microprojection array comprising: applying a vaccine antigen in a formulation to at least one microprojection, wherein the amount is applied such that the antigen dries on the projection in about 5 ms to about 5 seconds, and wherein the antigen the decrease in antigen potency is less than 5% after storage of the antigen at 4° C. for 1 month as to the dried antigen immediately after drying.

In one embodiment, the decrease in antigen potency is less than 10% after drying as compared to the antigen in solution.

In one embodiment, the decrease in antigen potency is less than 20% after drying as compared to the antigen in solution.

In one embodiment, the decrease in antigen potency is less than 30% after drying as compared to the antigen in solution.

In one embodiment, the formulation comprises at least one excipient.

In one embodiment, the antigen is an influenza HA antigen.

In one embodiment, the excipient is sulfobutyl ether β-cyclodextrin in an amount of from about 0.5% to about 5.0%.

In one embodiment, the antigen is an IPV antigen.

In one embodiment, the excipient is 4.5% SBE β-Cyclodextrin and 15 mM Glutathione.

In one embodiment, the excipient is 2.5% γ-Cyclodextrin and 15 mM Glutathione.

In one embodiment, the antigen potency is determined by ELISA.

In another broad form an aspect of the present invention seeks to provide a method of coating vaccine antigens onto a plurality of microprojections on a microprojection array comprising: applying a first amount of a first antigen to a first microprojection, wherein the amount is applied such that the first antigen dries on the projection in from about 5 ms to about 5 seconds; and applying a second amount of a second antigen to a second microprojection, wherein the amount is applied such that the second antigen dries on the projection in from about 5 ms to about 5 seconds, and wherein the second microprojection is directly adjacent the first microprojection, and wherein the second microprojection is about 10 to 200 μm from the first microprojection.

In one embodiment, the first antigen and second antigen are applied using an aseptic device rapid jetting device.

In another broad form an aspect of the present invention seeks to provide a method of coating materials onto a surface comprising: applying a first amount of a first material to a first feature on the surface, wherein the amount is applied such that the first material dries on the projection in from about 5 ms to about 5 seconds; and applying a second amount of a second material to a second feature on the surface, wherein the amount is applied such that the second material dries on the projection in from about 5 ms to about 5 seconds, and wherein the second feature is directly adjacent the first feature, and wherein the second feature is about 10 to 200 μm from the first feature.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:—

FIGS. 1A and 1B are plots of lead excipient concentration optimization with A/California/07/2009 MPH in a DPBS base buffer in a dried state. Dried MPH with different excipient concentrations were incubated at 48° C. for 0, 7, 14, and 28 days. FIG. 1A plots BCA determined protein recovery on the γ-axis and FIG. 1B plots EIA determined HA potency on the γ-axis. Each condition (for both protein recovery and HA potency) is shown as a relative percentage to a Day 0 sonicated stock solution. All error bars represent the standard deviation from quadruplicate experiments.

FIGS. 2A and 2B are plots of excipient combination screen with A/California/07/2009 MPH in a DPBS base buffer in a dried state. Dried MPH with different excipient concentrations were incubated at 48° C. for 0, 7, 14, and 28 days. FIG. 2A plots BCA determined protein recovery on the γ-axis and FIG. 2B plots EIA determined HA potency on the γ-axis. Each condition (for both recovery and potency) is shown as a relative percentage to a Day 0 sonicated stock solution. All error bars represent the standard deviation from quadruplicate experiments.

FIGS. 3A-3E are plots of results of a tIPV stability study monitoring D-antigen potency loss with top two candidate formulations during drying, storage for two weeks, and total potency loss. Potency loss for each of three IPV serotype in candidate formulations and in no excipient M199/DPBS control during drying is represented in FIG. 3A, during 4° C. and 25° C. storage for 2 weeks is represented in FIG. 3B, and FIGS. 3C-3E represent total potency loss after drying and storage. Data points are means with error bars representing 1SD from triplicate experiments.

FIGS. 4A-4C are plots of potency loss of dried tIPV in a M199/DPBS base buffer with various excipient combinations after 3 months of incubation at 4° C. Total potency loss for each of three IPV serotypes IPV1 (FIG. 4A), IPV2 (FIG. 4B), and IPV3 (FIG. 4C) in excipient combinations listed in Table 2.9 after 4° C. storage for 3 months. Excipients tested: β-CD: 4.5% w/v SBE-β-cyclodextrin; Glut: 15 mM glutathione; His: 30 mM histidine; γ-CD: 2.5% w/v γ-cyclodextrin; Arg: 0.15 M arginine; Cys: 20 mM cysteine. Data points are means with error bars representing 1SD from triplicate experiments. (Please refer to method section 1.2.6 for potency loss calculation).

FIG. 5A is a plot of HA concentration and protein content versus various time points for A/Singapore in 1% polyvinylpyrrolidone on LCP discs at 2-8° C.; FIG. 5B is a plot of HA concentration and protein content versus various time points for A/Singapore 3% arginine on LCP discs at 2-8° C. and FIG. 5C is a plot of HA concentration and protein content versus various time points for A/Singapore in 0.9% arginine and 0.3% SBECD on LCP discs at 2-8° C.

### DETAILED DESCRIPTION OF THE PREFERRED EMBODIMENTS

The present invention relates to devices and methods for coating microprojection or microneedle arrays with various substances. These substances may be liquid or non-liquid and may be coated onto the microprojection array such that one substance may be coated onto one microprojection and another substance may be coated onto a different microprojection. The present invention also relates to microprojection arrays having a base and a plurality of microprojections where the microprojections are divided into at least different sections or areas where each section or area has a plurality of microprojections and where the microprojections in one of the sections or areas are coated with one substance and where the microprojections in another area or section are coated with a different substance.

Arrays as used herein refers to devices that include one or more structures such as microprojections capable of piercing the stratum corneum to facilitate transdermal delivery of therapeutic agents through or to the skin.

Microprojections, as used herein, refers to the specific microscopic structures associate with the array that are capable of piercing the stratum corneum to facilitate transdermal delivery of therapeutic agents through or to the skin. Microprojections may include needle or needle-like structures, micro-pins as well as solid projections.

Microprojection and microneedle arrays can be in the form of patch having projections extending from a surface of a base. The projections and base may be formed from any suitable material, including but not limited to silicon and various polymers. The projections may be solid, non-porous and non-hollow as well as porous and/or hollow. Porous and/or hollow projections may be used to increase the volume of coating that can be accommodated on each projection such that coating is contained in pores or hollow portions of the projections. In such cases the material may be delivered over time as the coating on the outer surface of the patch dissolves first, with coating in the pores dissolving subsequently when the outer coating has dissolved and the pores are exposed to the surrounding tissues. Hollow projections can also be used for delivery of non-liquid coatings.

In an array the patch has a width W and a breadth B with the projections being separated by spacing. The projections may be provided in an array that is defined by a regular iteration of microprojections along a square or rectangular arrangement, but other arrangements of projections such as circular arrangement of the projections that are compatible with rotational spray coating may also be used. In order to further improve or enhance the targeting accuracy, the substrate may be designed such that the features to be coated are located on radial lines from the center point of the rotation or located on concentric circles or on a continuous spiral. The substrate may be designed such that the feature spacing on each arc is designed to match an integer number of steps of the motor for a given radius. Each projection includes a tip for penetrating tissue of the biological subject and projections will typically have a profile which tapers from the base to the tip.

The patch is applied to the biological subject by positioning the patch against a surface of a subject or by positioning the patch near the subject if an applicator that can propel the patch toward the skin is utilized. The tips of the projections penetrate the surface of the skin and may penetrate tissue beneath the surface of the skin to a given depth as the patch is applied. The patch may be used to deliver material or stimulus to internal tissues of a patient. The patch may be delivered such that the projections pierce the Stratum Corneum SC, and penetrate through the Viable Epidermis VE to penetrate the Dermis DE by a dermal penetration depth. The patch may be used to deliver material or stimulus to any part or region in the subject. The patch can be provided in a variety of different configurations to suit different material or stimulus delivery requirements. Accordingly, the specific configuration of the patch can be selected to allow the delivery of material and stimulus to particular tissues, at a specific depth, to induce a desired response.

The microprojection arrays that the applicator of the present invention projects into the skin may have a variety of shapes and sizes. The microprojection array may be square, circular, rectangular or irregular depending on its use. In some embodiments the microprojection arrays are square and have an equal number of microprojections in each row. For example the microprojection array may have 10 rows of 10 microprojections for a 10×10 array of 100 microprojections or 20 rows of 20 microprojections for a 20×20 array of 400 microprojections or 30 rows of 30 microprojections for a 30×30 array of 900 microprojections or 40 rows of 40 microprojections for a 40×40 array of 1600 microprojections or 50 rows of 50 microprojections for a 50×50 array of 2500 microprojections or 60 rows of 60 microprojections for a 60×60 array of 3600 or 70 rows of microprojections for a 70×70 array of 4900 microprojections or 80 rows of 80 microprojections for a 80×80 array of 6400 microprojections or 90 rows of 90 microprojections for a 90×90 array of 8100 or 100 rows of 100 microprojections for a 100×100 array of 10000 microprojections. The microprojection arrays may be in the shape of a rectangle where the number of rows does not equal the number of microprojections in a row. For example the microprojection array may have 10 rows of 20 microprojections for a 10×10 array of 200 microprojections or 20 rows of 30 microprojections for a 20×30 array of 600 microprojections or 30 rows of 40 microprojections for a 30×40 array of 1200 microprojections or 40 rows of 50 microprojections for a 40×50 array of 2000 microprojections or 50 rows of 60 microprojections for a 50×60 array of 3000 microprojections.

The microprojection arrays may be divided into areas such that a different vaccine antigen or other substance such as an excipient may be coated in each area. For example, the microprojection array may be divided in half or into four equal quadrants where different vaccine antigens or other substances such as excipients may be applied. These areas may have equal numbers of microprojections or unequal numbers of microprojections. In other embodiments some of the microprojections may be uncoated. For example a microprojection array having 80 rows of 80 projections for a total of 6400 microprojections may be divided into two equal sections of 3200 microprojections where 3200 microprojections are coated with a measles vaccine and the other 3200 microprojections are coated with a mumps vaccine. Alternatively the microprojection array can be divided into any number of areas including 2, 3, 4, 5, 6, 7, 8, 9 or 10 areas or more. Each microprojection in each area may be coated with a different substance. While the number of microprojections in an area can be between 1 and 20,000, the number of microprojection in an area should be sufficient to be coated with enough vaccine to make an effective dose of vaccine. Thus, the number of microprojections in an area may be 500 or more or 1000 or more or 2000 or more or 3000 or more or 4000 or more or 5000 or more or 6000 or more or 7000 or more or 8000 or more or 9000 or more or 10000 or more or 15000 or more. The number of microprojections in an area may be between 500 to 15000 or 500 to 10000 or 500 to 5000 or 500 to 4000 or 500 to 3000 or 500 to 2000 or 500 to 1000 or 1000 to 15000 or 1000 to 10000 or 1000 to 5000 or 1000 to 4000 or 1000 to 3000 or 1000 to 2000 or 2000 to 15000 or 2000 to 10000 or 2000 to 5000 or 2000 to 4000 or 2000 to 3000 or 3000 to 15000 or 3000 to 10000 or 3000 to 5000 or 3000 to 4000.

The microprojection arrays can be varied in size depending on its use. The area of the patch will have an impact on the ability to penetrate the subject, but this must be balanced by the need to induce cell damage over a sufficiently large area to induce a response. Consequently the patches typically have dimensions of between 0.5×0.5 mm and 20×20 mm, between 0.5×0.5 mm and 15×15 mm and more typically between 1×1 mm and 10×10 mm.

In one embodiment the microprojection array is 10×10 mm. The microprojection arrays may have a density of projections of between 1,000 to 20,000 per cm² or from 1,000 to per cm², or from 1,000 to 10,000 per cm² for from 1,000 to 5,000 per cm², or from 2,500 to 20,000 per cm² or from 2,500 to 15,000 per cm² or from 2,500 to 10,000 per cm² or from 2,500 to 7,500 per cm² or from 2,500 to 5,000 per cm² or from 5,000 to 20,000 per cm² or from 5,000 to 15,000 per cm² or from 5,000 to 10,000 per cm² or from 5,000 to 9,000 per cm² or from 5,000 to 8,000 per cm² or from 5,000 to 7,000 per cm² or from 5,000 to 6,000 per cm². The applicators of the present invention are often utilized to project high density microprojection arrays into the skin. Such high density arrays are microprojection arrays of sufficient size and density such that forces that can be applied manually will be insufficient to overcome the elasticity of the skin. The projections are typically separated by between 10 μm and 200 μm, between 30 μm and 150 μm, between 50 μm and 120 μm and more typically between 70 μm and 100 μm, leading to patches having between 10 and 1000 projections per mm² and more typically between 100 and 3000 projections per mm², and in one specific example approximately 20,000 per cm².

The length of the projections may be from 100 μm to 700 μm or from 100 μm to 600 μm or from 100 μm to 500 μm or from 100 μm to 400 μm or from 100 μm to 300 μm or from 100 μm to 250 μm or from 100 μm to 200 μm or from 150 μm to 700 μm or from 150 μm to 600 μm or from 150 μm to 500 μm or from 150 μm to 400 μm or from 150 μm to 300 μm or from 150 μm to 250 μm or from 150 μm to 200 μm or from 200 μm to 700 μm or from 200 μm to 600 μm or from 200 μm to 500 μm or from 200 μm to 400 μm or from 200 μm to 300 μm or from 200 μm to 250 μm or from 225 μm to 700 μm or from 225 μm to 600 μm or from 225 μm to 500 μm or from 225 μm to 400 μm or from 225 μm to 300 μm or from 225 μm to 250 μm or from 250 μm to 700 μm or from 250 μm to 600 μm or from 250 μm to 500 μm or from 250 μm to 400 μm or from 250 μm to 300 μm.

The projections may have a step shoulder (discontinuity) between the cone and pillar of the projection. In the event that a discontinuity is provided, this is typically located so that as the discontinuity reaches the dermis, penetration of the projection stops, with the tip extending into the dermal layer. Typically the discontinuity is located from the end of the tip at between 50 and 200 μm, between 50 and 190 μm, between 50 and 180 μm, between 50 and 170 μm, between 50 and 160 μm, between 50 and 150 μm, between 50 and 140 μm, between and 130 μm, between 50 and 120 μm, between 50 and 110 μm, between 50 and 100 μm, between 50 and 90 μm, between 50 and 80 μm, 60 and 200 μm, between 60 and 190 μm, between 60 and 180 μm, between 60 and 170 μm, between 60 and 160 μm, between 60 and 150 μm, between 60 and 140 μm, between 60 and 130 μm, between 60 and 120 μm, between and 110 μm, between 60 and 100 μm, between 60 and 90 μm, between 60 and 80 μm, 70 and 200 μm, between 70 and 190 μm, between 70 and 180 μm, between 70 and 170 μm, between 70 and 160 μm, between 70 and 150 μm, between 70 and 140 μm, between 70 and 130 μm, between 70 and 120 μm, between 70 and 110 μm, between 70 and 100 μm, between and 90 μm, between 70 and 80 μm, between 80 and 200 μm, between 80 and 190 μm, between 80 and 180 μm, between 80 and 170 μm, between 80 and 160 μm, between 80 and 150 μm, between 80 and 140 μm, between 80 and 130 μm, between 80 and 120 μm, between and 110 μm, between 80 and 100 μm, between 80 and 90 μm, between 90 and 200 μm, between 90 and 190 μm, between 90 and 180 μm, between 90 and 170 μm, between 90 and 160 μm, between 90 and 150 μm, between 90 and 140 μm, between 90 and 130 μm, between and 120 μm, between 90 and 110 μm, between 90 and 100 μm, between 100 and 200 μm, between 100 and 190 μm, between 100 and 180 μm, between 100 and 170 μm, between 100 and 160 μm, between 100 and 150 μm, between 100 and 140 μm, between 100 and 130 μm, between 100 and 120 μm, between 100 and 110 μm. The discontinuity may provide for greater loading of the drug/vaccine/excipient onto the microprojection.

The microprojection array may be made of any suitable materials including but not limited to metals, silicon, polymers, and plastic. In silicon embodiments the base thickness is about 60 μm or silicon with a thin (1 mm) polymer backing. The overall mass of some embodiments of the microprojection array is about 0.3 μm. The microprojection array may have bevelled edges to reduce peak stresses on the edge of the array. The patch can be quartered or subdivided by other ratios to reduce the stress load on the patch and mitigate patch breakage. Polymer embodiments may have reduced mass. The microprojection array may also have an overall weakly convex shape of the patch to improve the mechanical engagement with skin and mitigate the effect of high speed rippling application: a ‘high velocity/low mass’ system. The microprojection array may have a mass of less than 1 gram, or less than 0.9 grams or less than 0.8 grams or less than 0.7 grams, or less than 0.6 grams or less than 0.5 grams or less than 0.6 grams, or less than 0.5 grams or less than 0.4 grams or less than 0.3 grams or less than 0.2 grams or less than 0.1 grams or less than 0.05 grams. The microprojection array may have a mass of about 0.05 grams to about 2 grams, or from about grams to about 1.5 grams or from about 0.05 grams to about 1.0 grams or from about grams to about 0.9 grams, or from about 0.05 grams to about 0.8 grams or from about grams to about 0.7 grams, or from about 0.05 grams to about 0.6 grams or from about grams to about 0.5 grams or from about 0.05 grams to about 0.4 grams, or from about grams to about 0.3 grams or from about 0.05 grams to about 0.2 grams, or from about grams to about 0.1 grams or from about 0.1 grams to about 1.0 grams or from about 0.1 grams to about 0.9 grams, or from about 0.1 grams to about 0.8 grams or from about 0.1 grams to about 0.7 grams, or from about 0.1 grams to about 0.6 grams or from about 0.1 grams to about 0.5 grams or from about 0.1 grams to about 0.4 grams, or from about 0.1 grams to about 0.3 grams or from about 0.1 grams to about 0.2 grams. In one embodiment of the applicator/microprojection system the mass of the array is about 0.3 grams, the array is projected at a velocity of about 20-26 m/s by the applicator.

The projection spacing is selected so that material from the projections is able to, at least partially, provide spacing such that each individual projection can be coated separately. Accordingly, the projections are typically separated by between 10 μm and 200 μm or between 10 μm and 190 μm or between 10 μm and 180 μm or between 10 μm and 170 μm or between 10 μm and 160 μm or between 10 μm and 150 or between 10 μm and 140 μm or between 10 μm and 130 μm or between 10 μm and 120 μm or between 10 μm and 110 μm or between 10 μm and 100 μm or between 10 μm and 90 μm or between 10 μm and 80 μm or between 10 μm and 70 μm or between 10 μm and 60 μm or between 10 μm and 50 μm or between 10 μm and 40 μm or between 10 μm and 30 μm or between 10 μm and 20 μm or between 20 μm and 200 μm or between 20 μm and 190 μm or between 20 μm and 180 μm or between 20 μm and 170 μm or between 20 μm and 160 μm or between 20 μm and 150 or between 20 μm and 140 μm or between 20 μm and 130 μm or between 20 μm and 120 μm or between 20 μm and 110 μm or between 20 μm and 100 μm or between 20 μm and 90 μm or between 20 μm and 80 μm or between 20 μm and 70 μm or between 20 μm and 60 μm or between 20 μm and 50 μm or between 20 μm and 40 μm or between 20 μm and 30 μm or between 30 μm and 200 μm or between 30 μm and 190 μm or between 30 μm and 180 μm or between 30 μm and 170 μm or between 30 μm and 160 μm or between 30 μm and 150 or between 30 μm and 140 μm or between 30 μm and 130 μm or between 30 μm and 120 μm or between 30 μm and 110 μm or between 30 μm and 100 μm or between 30 μm and 90 μm or between 30 μm and 80 μm or between 30 μm and 70 μm or between 30 μm and 60 μm or between 30 μm and 50 μm or between 30 μm and 40 μm or between 40 μm and 200 μm or between 40 μm and 190 μm or between 40 μm and 180 μm or between 40 μm and 170 μm or between 40 μm and 160 μm or between 40 μm and 150 or between 40 μm and 140 μm or between 40 μm and 130 μm or between 40 μm and 120 μm or between 40 μm and 110 μm or between 40 μm and 100 μm or between 40 μm and 90 μm or between 40 μm and 80 μm or between 40 μm and 70 μm or between 40 μm and 60 μm or between 40 μm and 50 μm or between 50 μm and 200 μm or between 50 μm and 190 μm or between 50 μm and 180 μm or between 50 μm and 170 μm or between 50 μm and 160 μm or between 50 μm and 150 or between 50 μm and 140 μm or between 50 μm and 130 μm or between 50 μm and 120 μm or between 50 μm and 110 μm or between 50 μm and 100 μm or between 50 μm and 90 μm or between 50 μm and 80 μm or between 50 μm and 70 μm or between 50 μm and 60 μm or between 60 μm and 200 μm or between 60 μm and 190 μm or between 60 μm and 180 μm or between 60 μm and 170 μm or between 60 μm and 160 μm or between 60 μm and 150 or between 60 μm and 140 μm or between 60 μm and 130 μm or between 60 μm and 120 μm or between 60 μm and 110 μm or between 60 μm and 100 μm or between 60 μm and 90 μm or between 60 μm and 80 μm or between 60 μm and 70 μm or between 70 μm and 200 μm or between 70 μm and 190 μm or between 70 μm and 180 μm or between 70 μm and 170 μm or between 70 μm and 160 μm or between 70 μm and 150 or between 70 μm and 140 μm or between 70 μm and 130 μm or between 70 μm and 120 μm or between 70 μm and 110 μm or between 70 μm and 100 μm or between 70 μm and 90 μm or between 70 μm and 80 μm or between 80 μm and 200 μm or between 80 μm and 190 μm or between 80 μm and 180 μm or between 80 μm and 170 μm or between 80 μm and 160 μm or between 80 μm and 150 or between 80 μm and 140 μm or between 80 μm and 130 μm or between 80 μm and 120 μm or between 80 μm and 110 μm or between 80 μm and 100 μm or between 80 μm and 90 μm or between 90 μm and 200 μm or between 90 μm and 190 μm or between 90 μm and 180 μm or between 90 μm and 170 μm or between 90 μm and 160 μm or between 90 μm and 150 or between 90 μm and 140 μm or between 90 μm and 130 μm or between 90 μm and 120 μm or between 90 μm and 110 μm or between 90 μm and 100 μm or between 100 μm and 200 μm or between 100 μm and 190 μm or between 100 μm and 180 μm or between 100 μm and 170 μm or between 100 μm and 160 μm or between 100 μm and 150 or between 100 μm and 140 μm or between 100 μm and 130 μm or between 100 μm and 120 μm or between 100 μm and 110 μm.

In some embodiments, more than one coating may be applied to the same projection. For instance, different coatings may be applied in one or more layers to provide the same or different materials for delivery to the tissues within the subject at the same time or different times if the layers dissolve in sequence. A first coating may be applied to modify surface properties of the projection and improve the ability of the second coating to coat the projection in a desirable manner. Multiple layers of the same coating formulation may be used with drying between each layer to allow a progressive build up of coating to achieve a specific thickness and thus modify the effective cross section of the projection even further. A layer of one substance may be applied to the microprojection which may then be subsequently coated with a second substance. It may also be possible to coat the microprojection with a single substance multiple times to form multiple layers of the one substance and then apply multiple layers of a second substance over the layers of the first substance. More than two substances may be applied to the same microprojection. The first substance may be applied to the microprojection is such a manner that the application of a second substance to the same microprojection completely overcoats, partially overcoats or does not overcoat the first substance applied to the microprojection. Substances may be applied to the microprojections in such a manner that multiple substances are located at different portions of the microprojection after coating. For example, substances may be applied to the microprojections such that a first substance is coated at the bottom of the microprojection and a second substance is coated at the top (tip) of the microprojection. Substances may be applied to the microprojections such that a first substance is coated on one side of the microprojection and a second substance is coated on the other side of the microprojection. In certain embodiments of the patches of the present invention the patch may be divided into sections in which each of the microprojections within that section are coated with identical substances but each of the sections has a different substance on its microprojections.

Substances applied to the microprojections can be of various types including but not limited to small chemical or biochemical compounds including antigens, ligands, drugs, metabolites, amino acids, sugars, lipids, saponins, and hormones; macromolecules such as complex carbohydrates, phospholipids, peptides, polypeptides, proteins, peptidomimetics, and nucleic acids; or other organic (carbon containing) or inorganic molecules; and particulate matter including whole cells, bacteria, viruses, virus-like particles, cell membranes, dendrimers and liposomes or combinations thereof. Substances may also include contrast enhancing reagents or surface modifying materials.

The substances may be comprised of a single compound or multiple compounds. For example, in embodiments used for vaccination the microprojections may be coated with a vaccine compound that contains a single antigen or multiple antigens either to the same pathogen or to different pathogens. In another embodiment the substance may be a vaccine composition having an excipient and one or more antigens. In another embodiment the substance may be a vaccine composition having an adjuvant and one or more antigens As described above vaccine compositions may be delivered by the patch such that different antigens are located on different microprojections either independent one from another or in sections located on the patch. For example, antigens for measles might be on one section of the patch and antigens for mumps and rubella on different sections of the patch. Or the antigens for each measles, mumps and rubella on different individual microprojections within the patch. Vaccine compositions may be delivered by the patch such that one or more antigens are located on different microprojections and adjuvants and/or excipients are independent one from another. In another embodiment the microprojection array may be partitioned into sections such that each section of the array has microprojections covered with a different substance. For example one section of the microprojection array might contain microprojections covered with an adjuvant while other sections of the array might contain microprojections coated with antigens. Alternatively, one section of the microprojection array might contain microprojections coated with a substance that contain an antigen and an adjuvants while another section of the microprojection array contains microprojections coated with a different antigen than the first section either with or without an adjuvant. Such designs that place different substances on different sections of the patch or on different microprojections are useful when the substances are incompatible. Some multivalent vaccine formulations can contain antigens and/or excipients which are not compatible. In such cases the ability to place the antigens and excipients on different microprojections may help reduce the incompatibility of the antigens, excipients and/or adjuvants. The challenge of providing combination vaccines with multiple valencies and adjuvants is described in Skibinski et al. (2011) J. of Global Infectious Disease January-March 3(1): 63-72.

Coatings may be liquid or non-liquid. Liquid coating materials may aqueous, however other coating solutions are possible, and that the surface properties of the projection may need to be modified to accommodate a range of coating solutions. For an aqueous coating solution, the microprojections may be modified to be more “hydrophobic” in nature. A hydrophilic surface will cause an aqueous solution to completely wet it (assuming low viscosity). This would result in a large fraction of the liquid coating material being wicked onto the base of the projection array, which would impede its delivery to the skin. Increasing the solution viscosity slows down the wicking (or surface wetting) process. If a dry coating process is accomplished rapidly in comparison to the surface wetting, a larger fraction of the liquid coating material can be localized to the projections. By changing the contact angle of the projection surface (by chemically modifying it), the liquid coating solution wetting properties may also be altered. In making the surface more “hydrophobic”, an aqueous coating solution will be inhibited from wetting the projection surface down to the base. Furthermore, a surfactant can be added to an aqueous coating solution which is placed on a “hydrophobic” projection. The surfactant may assist in wetting the hydrophobic surface by orienting the polar and non-polar groups of the surfactant at the surface, thus facilitating the wetting. If appropriate drying conditions (either with or without surfactant) are achieved, the result is that a significant portion of the coating material is retained near the projection tips. Striking a balance between the surface wetting properties (i.e. contact angle), solution viscosity, and the presence or absence of a surfactant (among other solution properties) can change the degree and uniformity with which the coating solution is localized to the projection tips. In a further embodiment, the microprojection surface may be altered such that the tips are hydrophilic and the lower portion of the shaft and base are hydrophobic. This can be accomplished using bulk lithographic processes. In this embodiment, the hydrophilic tip surface is easily wet, while the lower portion of the projection inhibits liquid travel towards the base due to its hydrophobic nature. Other methods of coating the microprojections include but are not limited to differential coatings using plasma polymers, spin coating, microimprinting and dip coating.

The vaccines employed in the present invention may contain live, attenuated, modified or killed microorganisms or their toxins or tumor antigens which when administered into the body stimulate the body's immune system to produce antigen-specific antibodies.

Some of the substances utilized for delivery by the microprojections include antigens from pathogenic organisms which include, but are not limited to, viruses, bacteria, fungi, parasites, algae and protozoa and amoebae. Illustrative viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen/vaccine derived from such viruses is useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, representative fungal antigens that can be used in the compositions and methods of the present invention include, but are not limited to, candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae.), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487). Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components (such as Group A strep antigen); gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumoly sin, pneumococcal capsular polysaccharides and other pneumococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS 198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

Also included are DNA and RNA antigens. The presentation of the antigen and particulate form-lipid nanoparticle encapsulated, Virus like particles, conjugated (protein or polysaccharide) etc.

The amount of antigen used in the devices and methods of the present invention include amounts necessary to provide an immune response. At least one dose selected from the group consisting of a 1 μg dose, 2 μg dose, 3 μg dose, 4 μg dose, 5 μg dose, 6 μg dose, 7 μg dose, 8 μg dose, 9 μg dose, 10 μg dose, 15 μg dose, 20 μg dose, 25 μg dose, a 30 μg dose, 40 μg dose, 50 μg dose, 60 μg dose, 70 μg dose, 80 μg dose, 90 μg dose, 100 μg dose, 125 μg dose, 150 μg dose, 200 μg dose, 250 μg dose, 300 μg dose, 350 μg dose, 400 μg dose per antigen, may be sufficient to induce an immune response in humans. The dose of each antigen may be administered to the human within a range of doses including from about 1 μg to about 50 μg, from about 1 μg to about 30 μg, from about 1 μg to about 25 μg, from about 1 μg to about 20 μg, from about 1 μg to about 15 μg, from about 1 μg to about 10 μg, from about 2 μg to about 10 μg, from about 2 μg to about 8 μg, from about 3 μg to about 10 μg, from about 3 μg to about 8 μg, from about 3 μg to about 5 μg, from about 4 μg to about 10 μg, from about 4 μg to about 8 μg, from about 5 μg to about 10 μg, from about 5 μg to about 9 μg, and from about 5 μg to about 8 μg. For example, HPV has 270 ug of antigen (albeit 9 different HPV types), Hib has 132.5 ug (PRP+OMPC conjugate). Including the excipients that may be necessary as part of the vaccine, this typically brings the total solids into the milligram range e.g. Flu dose is >4 mg, polio is above 7 mg, Hib is above 4 mg, MMRII is above 30 mg.

The present invention also relates to devices, formulations and methods for increasing the stability of vaccine formulations including but not limited to influenza and inactivated polio vaccine due to the use of excipients which include but are not limited to cyclodextrins, amino acids, reducing agents carbohydrates and proteins and combinations thereof. Excipients include but are not limited to Histidine, Sodium acetate, Sodium chloride, Sodium citrate, Sodium phosphate, Sodium sulfate, Sodium succinate, Gelatin, Hydrolysed Gelatin, Protamine sulfate, Arginine, Aspartic acid (sodium salt), Glutamic acid, Glycine, Isoleucine, Lactic acid, Lysine, Maleic acid, Malic acid (sodium salt), Methionine, Urea, EDTA, Magnesium chloride, Benzalkonium chloride, Brij 35, Poloxamer 188 (Pluronic F-68), Polysorbate 20, Polysorbate 80, Sodium docusate, Triton X-100, Lactose, Sucrose, Trehalose, Glycerol, Mannitol, Sorbitol, Gamma-Cyclodextrin, 2-OH propyl b-CD, Sulfobutyl ether beta-cyclodextrin, Carboxymethyl cellulose, Dextran sulfate, Dextran 40, PEG-3350, Sodium Hyaluronate, Sodium thioglycolate, Cysteine, and Glutathione and combinations thereof.

In some cases a vaccine adjuvant may be necessary to enhance the vaccine's ability to induce protection against infection. Adjuvants help activate the immune system, allowing the antigens-pathogens components that elicit an immune response in vaccines to induce long-term protective immunity. Adjuvants include but are not limited to pathogen components such as monophosphoryl lipid A (which has been combined with alum to produce AS04), poly(I:C) (which is a synthetic double stranded RNA), CpG DNA adjuvants (which are short segments of DNA) and emulsions such as MF59 which is an oil in water emulsion that include squalene and AS03 which is D,L-alpha-tocopherol (Vitamin E), an emulsifier, polysorbate 80 and squalene. Other adjuvants include particulate adjuvants such as alum, virosomes and cytokines.

The biological, immunological and physiochemical properties of antigens can be verified by a wide range of tests including but not limited to Western blot, epitope scanning, immunogenicity in mice, SDS-PAGe, MALDI?MS, transmission electron microscopy, isopynic gradient ultracentrifugation, dynamic light scattering, peptide mapping and amino acid sequencing. Stability of vaccine compositions and components can be measured by a loss in antigen activity such as potency. This loss in potency can be determined under a variety of conditions, such as storage temperature and storage humidity at various time points. Typically vaccines which are in solution are stored at 4° C. or at room temperature (about 25° C.). It would be preferable to be able to store vaccine at at least room temperature or higher temperatures (35° C.-45° C.) such that cold storage would be unnecessary. Quantification of hemagglutinin (HA) can be measured by single radial diffusion or other techniques such as HPLC, mass spectroscopy, ELISA and antibody dependent surface plasmon resonance (P. D. Minor (2015) Assaying the Potency of Influenza Vaccine, Vaccines 3, 90-104.

The methods and compositions of the present invention provide microprojection arrays that can be coated with multiple incompatible vaccine antigens that are stable over time. The vaccine compositions of the present invention are stable at at least 4° C. for at least 1 or at least 2 or at least 3 or at least 4 or at least 5 or at least 6 or at least 7 or at least 8 or at least 9 or at least 10 or at least 12 or at least 13 or at least 14 or at least 15 or at least 16 or at least 17 or at least 18 or at least 19 or at least 20 or at least 21 or at least 22 or at least 23 or at least 24 or at least 30 or at least 36 months at various temperatures and conditions. The stability of the vaccine formulations may be measured by a variety of techniques including but not limited to ELISA and SDS-PAGE silver stain.

The methods and compositions of the present invention provide microprojection arrays that can be coated with multiple incompatible vaccine antigens that are stable over time. The vaccine compositions of the present invention are stable at at least 25° C. for at least 1 or at least 2 or at least 3 or at least 4 or at least 5 or at least 6 or at least 7 or at least 8 or at least 9 or at least 10 or at least 12 or at least 13 or at least 14 or at least 15 or at least 16 or at least 17 or at least 18 or at least 19 or at least 20 or at least 21 or at least 22 or at least 23 or at least 24 or at least 30 or at least 36 months at various temperatures and conditions. The stability of the vaccine formulations may be measured by a variety of techniques including but not limited to ELISA and SDS-PAGE silver stain.

To evaluate vaccine stability following drying, antigen values of recovered vaccine were determined using the ELISA assay. The percent potency of recovered dried vaccine was calculated by normalizing the antigen values of recovered dried samples to the values of an in-liquid stock vaccine stored at 4° C., which was considered to have 100% potency. The drying potency loss was calculated by subtracting the percent potency of freshly dried vaccine samples (recovered immediately after drying) from the in-liquid stock vaccine stored at 4° C. (i.e., 100%−relative percent potency after drying=drying potency loss). Similarly, the storage potency loss was determined by subtracting the relative potency of the stored samples with the relative percent potency of the sample recovered immediately after drying (i.e., 100%−relative percent potency after storage−relative percent potency after drying=storage potency loss).

Reduction of potency for the formulations/antigens of the present invention upon rapid drying can be about 0% or less than about 5% or less than about 10% or less than about 15% or less than about 20% or less than about 25% or less than about 30% or less than about 35% or less than about 40% or less than about 45% or less than about 50% or less than about 55% or less than about 60% or less than about 65% or less than about 70% or less than about 75% or less than about 80% or less than about 85% or less than about 90%.

Reduction of potency for the formulations/antigens of the present invention upon rapid drying and storage at at least 4° C. for at least 1 or at least 2 or at least 3 or at least 4 or at least 5 or at least 6 or at least 7 or at least 8 or at least 9 or at least 10 or at least 12 or at least 13 or at least 14 or at least 15 or at least 16 or at least 17 or at least 18 or at least 19 or at least or at least 21 or at least 22 or at least 23 or at least 24 or at least 30 or at least 36 months can be about 0% or less than about 5% or less than about 10% or less than about 15% or less than about 20% or less than about 25% or less than about 30% or less than about 35% or less than about 40% or less than about 45% or less than about 50% or less than about 55% or less than about 60% or less than about 65% or less than about 70% or less than about 75% or less than about 80% or less than about 85% or less than about 90%.

Reduction of potency for the formulations/antigens of the present invention upon rapid drying and storage at at least 25° C. for at least 1 or at least 2 or at least 3 or at least 4 or at least 5 or at least 6 or at least 7 or at least 8 or at least 9 or at least 10 or at least 12 or at least 13 or at least 14 or at least 15 or at least 16 or at least 17 or at least 18 or at least 19 or at least 20 or at least 21 or at least 22 or at least 23 or at least 24 or at least 30 or at least 36 months can be about 0% or less than about 5% or less than about 10% or less than about 15% or less than about 20% or less than about 25% or less than about 30% or less than about 35% or less than about 40% or less than about 45% or less than about 50% or less than about 55% or less than about 60% or less than about 65% or less than about 70% or less than about 75% or less than about 80% or less than about 85% or less than about 90%.

In preferred embodiments the microprojections of the microprojection array are coated by an aseptic print-head type device which rapidly provides small droplets which dry quickly on the microprojections. In preferred embodiments the coating such as a vaccine formulation rapidly dries on the top portion of the microprojection to increase the amount of vaccine that can be delivered. The aseptic print head device may deliver multiple drops to the microprojections either sequentially or in an alternating fashion. In one embodiment of the print head device the device comprises the housing which is connected to the pumping chamber where the fluid to be dispensed is stored. The fluid flows into the pumping chamber through one or more ports. The unimorph piezoelectric device is activated and impinges on the plate membrane which is held by a restrictor plate. The descender plate is attached to the nozzle plate such that when the unimorph piezoelectric is activated, fluid is pushed by the plate membrane through the descender plate and out through the nozzles in the nozzle plate to be distributed onto the microprojections. The housing may have ports for conducting fluid into the pumping chamber. The unimorph PZT impacts the plate membrane which is held in place by a restrictor plate. All of these parts are assembled with the housing and the descender plate and nozzle plate. The embodiments utilizing the unimorph PZT are assembled using a bio-compatible epoxy.

Each drop ejection cycle enables all the nozzles to simultaneously dispense a drop or a sequence of drops with a total volume in the range of 10 to 1000 picoliters, or 10 to 900 picoliters, or 10 to 800 picoliters, or 10 to 700 picoliters, or 10 to 600 picoliters, or 10 to 500 picoliters, or 10 to 400 picoliters, or 10 to 300 picoliters, or 10 to 200 picoliters or 10 to 100 picoliters, 25 to 1000 picoliters, or 25 to 900 picoliters, or 25 to 800 picoliters, or 25 to 700 picoliters, or 25 to 600 picoliters, or 25 to 500 picoliters, or 25 to 400 picoliters, or 25 to 300 picoliters, or 25 to 200 picoliters or 25 to 100 picoliters, or 25 to 50 picoliters, or 75 to 1000 picoliters, or 75 to 900 picoliters, or 75 to 800 picoliters, or 75 to 700 picoliters, or 75 to 600 picoliters, or 75 to 500 picoliters, or 75 to 400 picoliters, or 75 to 300 picoliters, or 75 to 200 picoliters or 75 to 100 picoliters 100 to 1000 picoliters, or 100 to 900 picoliters, or 100 to 800 picoliters, or 100 to 700 picoliters, or 100 to 600 picoliters, or 100 to 500 picoliters, or 100 to 400 picoliters, or 100 to 300 picoliters, or 100 to 200 picoliters, or 200 to 1000 picoliters, or 200 to 900 picoliters, or 200 to 800 picoliters, or 200 to 700 picoliters, or 200 to 600 picoliters, or 200 to 500 picoliters, or 200 to 400 picoliters, or 200 to 300 picoliters, or 300 to 1000 picoliters, or 300 to 900 picoliters, or 300 to 800 picoliters, or 300 to 700 picoliters, or 300 to 600 picoliters, or 300 to 500 picoliters, or 300 to 400 picoliters, or 400 to 1000 picoliters, or 400 to 900 picoliters, or 400 to 800 picoliters, or 400 to 700 picoliters, or 400 to 600 picoliters, or 400 to 500 picoliters, or 500 to 1000 picoliters, or 500 to 900 picoliters, or 500 to 800 picoliters, or 500 to 700 picoliters, or 500 to 600 picoliters, or 600 to 1000 picoliters, or 600 to 900 picoliters, or 600 to 800 picoliters, or 600 to 700 picoliters, or 700 to 1000 picoliters, or 700 to 900 picoliters, or 700 to 800 picoliters or 800 to 1000 picoliters, or 800 to 900 picoliters, or 900 to 1000 picoliters. The drop size of each individual drop may be from about 100 to 200 picoliters, or 100 to 190 picoliters, or 100 to 180 picoliters, or 100 to 170 picoliters, or 100 to 160 picoliters, or 100 to 150 picoliters, or 100 to 140 picoliters, or 100 to 130 picoliters, or 100 to 120 picoliters or from 100 to 110 picoliters, or from about 110 to 200 picoliters, or 110 to 190 picoliters, or 110 to 180 picoliters, or 110 to 170 picoliters, or 110 to 160 picoliters, or 110 to 150 picoliters, or 110 to 140 picoliters, or 110 to 130 picoliters, or 110 to 120 picoliters or from about 120 to 200 picoliters, or 120 to 190 picoliters, or 120 to 180 picoliters, or 120 to 170 picoliters, or 120 to 160 picoliters, or 120 to 150 picoliters, or 120 to 140 picoliters, or 120 to 130 picoliters, or from about 130 to 200 picoliters, or 130 to 190 picoliters, or 130 to 180 picoliters, or 130 to 170 picoliters, or 130 to 160 picoliters, or 130 to 150 picoliters, or 130 to 140 picoliters, or from about 140 to 200 picoliters, or 140 to 190 picoliters, or 140 to 180 picoliters, or 140 to 170 picoliters, or 140 to 160 picoliters, or 140 to 150 picoliters, or from about 150 to 200 picoliters, or 150 to 190 picoliters, or 150 to 180 picoliters, or 150 to 170 picoliters, or 150 to 160 picoliters, or from about 160 to 200 picoliters, or 160 to 190 picoliters, or 160 to 180 picoliters, or 160 to 170 picoliters, or 170 to 200 picoliters, or 170 to 190 picoliters, or 170 to 180 picoliters, or 180 to 200 picoliters, or 180 to 190 picoliters or from 190 to 200 picoliters.

The frequency of dispensing the drops is from about 1 Hz to about 1000 Hz or from about 1 Hz to about 900 Hz or from about 1 Hz to about 800 Hz or from about 1 Hz to about 700 Hz or from about 1 Hz to about 600 Hz or from about 1 Hz to about 500 Hz or from about 1 Hz to about 400 Hz or from about 1 Hz to about 300 Hz or from about 1 Hz to about 200 Hz or from about 1 Hz to about 100 Hz or from about 1 Hz to about 90 Hz or from about 1 Hz to about or from about 1 Hz to about 70 Hz or from about 1 Hz to about 60 Hz or from about 1 Hz to about 50 Hz or from about 1 Hz to about 40 Hz or from about 1 Hz to about 30 Hz or from about 1 Hz to about 20 Hz or from about 1 Hz to about 10 Hz or from about 10 Hz to about 100 Hz or from about 10 Hz to about 90 Hz or from about 10 Hz to about 80 Hz or from about to about 70 Hz or from about 10 Hz to about 60 Hz or from about 10 Hz to about 50 Hz or from about 10 Hz to about 40 Hz or from about 10 Hz to about 30 Hz or from about 10 Hz to about 20 Hz or from about 20 Hz to about 100 Hz or from about 20 Hz to about 90 Hz or from about 20 Hz to about 80 Hz or from about 20 Hz to about 70 Hz or from about 20 Hz to about or from about 20 Hz to about 50 Hz or from about 20 Hz to about 40 Hz or from about to about 30 Hz or from about 30 Hz to about 100 Hz or from about 30 Hz to about 90 Hz or from about 30 Hz to about 80 Hz or from about 30 Hz to about 70 Hz or from about 30 Hz to about 60 Hz or from about 30 Hz to about 50 Hz or from about 30 Hz to about 40 Hz or from about 40 Hz to about 100 Hz or from about 40 Hz to about 90 Hz or from about 40 Hz to about or from about 40 Hz to about 70 Hz or from about 40 Hz to about 60 Hz or from about to about 50 Hz or from about 50 Hz to about 100 Hz or from about 50 Hz to about 90 Hz or from about 50 Hz to about 80 Hz or from about 50 Hz to about 70 Hz or from about 50 Hz to about 60 Hz or from about 60 Hz to about 100 Hz or from about 60 Hz to about 90 Hz or from about 60 Hz to about 80 Hz or from about 60 Hz to about 70 Hz or from about 70 Hz to about 100 Hz or from about 70 Hz to about 90 Hz or from about 70 Hz to about 80 Hz or from about to about 100 Hz or from about 80 Hz to about 90 Hz or from about 90 Hz to about 100 Hz.

The drying time of each droplet may be from about 1 millisecond (ms) to about 5 seconds (s) or from about 1 ms to about 4 s or from about 1 ms to about 3 s or from about 1 ms to about 2 s or from about 1 ms to about 1 s or from about 1 ms to about 500 ms or from about 1 ms to about 250 ms or from about 1 ms to about 100 ms or from about 25 ms to about 5 s or from about 25 ms to about 3 s or from about 25 ms to about 2 s or from about 25 ms to about 1 s or from about 25 ms to about 500 ms or from about 25 ms to about 250 ms or from about 25 ms to about 100 ms or from about 50 millisecond (ms) to about 5 seconds (s) or from about 50 ms to about 4 s or from about 50 ms to about 3 s or from about 50 ms to about 2 s or from about 50 ms to about is or from about 50 ms to about 500 ms or from about 50 ms to about 250 ms or from about 50 ms to about 100 ms or from about 100 millisecond (ms) to about 5 seconds (s) or from about 100 ms to about 4 s or from about 100 ms to about 3 s or from about 100 ms to about 2 s or from about 100 ms to about is or from about 100 ms to about 500 ms or from about 100 ms to about 250 ms or from about 500 millisecond (ms) to about 5 seconds (s) or from about 500 ms to about 4 s or from about 500 ms to about 3 s or from about 500 ms to about 2 s or from about 500 ms to about is or from about is to about 5 seconds (s) or from about 1 s to about 4 s or from about 1 s to about 3 s or from about 1 s to about 2 s.

The microprojections of the array of the present invention may be of any shape including cylindrical or conical. Other geometries are also possible. The microprojection arrays may have substrate with a plurality of microprojections protruding from the substrate wherein the microprojections have a tapering hexagonal shape and comprise a tip and a base wherein the base has two substantially parallel sides with a slight draught angle of approximately 1 to 20 degrees up to a transition point at which point the angle increases to from about 20 degrees to about 70 degrees. A sharp blade-like tip will allow for enhanced penetration of the microprojections into the skin while also generating an enhanced localized cell death/bystander interaction in the skin with a different profile than conical microprojection arrays. In a preferred embodiment the microprojections are made of a polymer and are slightly blunted at the tip with shoulders near the tip on which the coating material may attach such that the coating material does not drip down the microprojection and onto the base of the microprojection array.

In the present invention the density of the microprojections is relatively high which means the microprojections are spaced relatively close together. The density of the microprojection on the microprojection arrays may be about 500 microprojections/cm², or about 1000 microprojections/cm², or about 1500 microprojections/cm², or about 2000 microprojections/cm², or about 2500 microprojections/cm², or about 3000 microprojections/cm², or about 3500 microprojections/cm², or about 4000 microprojections/cm², or about 4500 microprojections/cm², or about 5000 microprojections/cm², or about 5500 microprojections/cm², or about 6000 microprojections/cm², or about 6500 microprojections/cm², or about 7000 microprojections/cm², or about 7500 microprojections/cm², or about 8000 microprojections/cm², or about 8500 microprojections/cm², or about 9000 microprojections/cm², or about 9500 microprojections/cm², or about 10000 microprojections/cm², or about 11000 microprojections/cm², or about 12000 microprojections/cm², or about 13000 microprojections/cm², or about 14000 microprojections/cm², or about 15000 microprojections/cm², or about 16000 microprojections/cm², or about 17000 microprojections/cm², or about 18000 microprojections/cm², or about 19000 microprojections/cm², or about 20000 microprojections/cm². The density of the microprojection on the microprojection arrays may be from about 2000 to about 20000 microprojections/cm², or from about 2000 to about 15000 microprojections/cm², or from about to about 10000 microprojections/cm², or from about 2000 to about 9000 microprojections/cm², or from about 2000 to about 8000 microprojections/cm², or from about 2000 to about 7500 microprojections/cm², or from about 2000 to about 7000 microprojections/cm², or from about 2000 to about 6000 microprojections/cm², or from about 2000 to about 5000 microprojections/cm², or from about 2000 to about 4000 microprojections/cm², or from about 3000 to about 20000 microprojections/cm², or from about 3000 to about 15000 microprojections/cm², or from about to about 10000 microprojections/cm², or from about 3000 to about 9000 microprojections/cm², or from about 3000 to about 8000 microprojections/cm², or from about 3000 to about 7500 microprojections/cm², or from about 3000 to about 7000 microprojections/cm², or from about 3000 to about 6000 microprojections/cm², or from about 3000 to about 5000 microprojections/cm², or from about 3000 to about 4000 microprojections/cm², or from about 4000 to about 20000 microprojections/cm², or from about 4000 to about 15000 microprojections/cm², or from about to about 10000 microprojections/cm², or from about 4000 to about 9000 microprojections/cm², or from about 4000 to about 8000 microprojections/cm², or from about 4000 to about 7500 microprojections/cm², or from about 4000 to about 7000 microprojections/cm², or from about 4000 to about 6000 microprojections/cm², or from about 4000 to about 5000 microprojections/cm², or from about 5000 to about 20000 microprojections/cm², or from about 5000 to about 15000 microprojections/cm², or from about to about 10000 microprojections/cm², or from about 5000 to about 9000 microprojections/cm², or from about 5000 to about 8000 microprojections/cm², or from about 5000 to about 7500 microprojections/cm², or from about 5000 to about 7000 microprojections/cm², or from about 5000 to about 6000 microprojections/cm².

### EXAMPLES

### Example 1

### Sample Preparation and Testing for Influenza Vaccine

Approximately 100 mL of A/California/07/2009 MPH vaccine stock (Lot #09061477200, containing 6.0 mg/mL hemagglutinin (HA) was provided in PBS (Phosphate-buffered saline) 10 mM Na₂HPO₄, 1.8 mM KH₂PO₄, 137 mM NaCl, 2.7 mM KCl, pH 7.2. This MPH stock solution was stored at 4° C. and used to develop stability-indicating methods. The formulation and concentration of the MPH vaccine stock are displayed below.

A 96 well drying rig (plus tubing), anti-A/California/01/05 monoclonal antibody, Horseradish peroxidase (HRP)-conjugated anti-A/California/01/05 monoclonal antibody, A/California/07/2009 standards for enzyme immunoassay, Vaxigrip 2014 vaccine, 6 mm liquid crystal polymer (LCP) discs, trehalose and 6% w/w hypromellose solution were used in the example.

For in-solution samples stock MPH was diluted with an equal volume of DPBS (1:1) to make an in-solution sample containing 3.0 mg/mL HA. The samples were aliquoted into PCR tubes (20 μL/tube and 2 tubes/replicate).

For dried samples LCP discs were placed into TPP 96-well plate, 1 disc/well. MPH was diluted with equal volume of excipient or DPBS solution 1:1 (formulated MPH contains 3.0 mg/mL HA). 5 μl of formulated MPH was dispensed onto the center of each disc (15 μg HA/disc) by reverse pipetting. The plate was then transferred to the drying rig, and dried for 13 min under 14 L/min N2 flow. Then the plate was sealed with adhesive film.

In-solution and dried on-disc MPH samples were incubated at different temperatures and for different durations, depending on the design of the stability study (see the Results section below for more details). Dried on disc samples in 96 well plates were sealed with thermo-stable film, and stored with desiccant.

Concentrated (2×) stock excipients were dissolved in DPBS (pH 7.2), the pH was then adjusted to 7.2, and the solution was sterilized by filtering through 0.22 μm PVDF membrane (the first 5 mL of each solution passing through the PVDF membrane was discarded to eliminate any potential contamination of residual particles or extractables from the filters). The excipient stock solutions were stored at 4° C. for up to 1 month (unstable excipient such as DTT solution were prepared immediately before use). The list of excipients used is shown in Table 1.

TABLE 1
List of excipients.

| # | Excipients | Supplier | Cat. # | Lot # |
|---|---|---|---|---|
| 1 | Histidine | Sigma-Aldrich | 53319-100G | BCBL8528V |
| 2 | Sodium acetate | Sigma-Aldrich | S2889-250G | SLBF5613V |
| 3 | Sodium chloride (salt) | Sigma-Aldrich | S6191-500g | 081M00941V |
| 4 | Sodium citrate | Sigma-Aldrich | W302600-1KG-K | MKBP1879V |
| 5 | Sodium phosphate | Sigma-Aldrich | S7907-100G | BCBK2922V |
| 6 | Sodium sulfate (salt) | Sigma-Aldrich | 238597-500G | MKBP7388V |
| 7 | Sodium succinate | Sigma-Aldrich | 14160-100G | BCBK0561V |
| 8 | Tris (TromeThamine) | Sigma-Aldrich | T1503-250G | SLBF3424V |
| 9 | Human Albumin | Sigma-Aldrich | A3782-100MG | SLBD7204V |
| 10 | Hydrolysed Gelatin | Sigma-Aldrich | G-7041 | 41K1575 |
| 11 | Protamine sulfate | Sigma-Aldrich | 3369-10G | SLBG6301V |
| 12 | Arginine | Sigma-Aldrich | A5006-100G | MKBG3766V |
| 13 | Aspartic acid | Sigma-Aldrich | 11195-100G | BCBM9719V |
| 14 | Glutamic acid | Fluka | 49621 | 1117485 |
| 15 | Glycine | Sigma-Aldrich | 8898-500G | SLBK4571V |
| 16 | Histidine | Sigma-Aldrich | 53319-100G | BCBL8528V |
| 17 | Isoleucine | Fluka | 58879 | 1113064 |
| 18 | Lactic acid | Sigma-Aldrich | 252476-100G | MKBB6991 |
| 19 | Lysine | Sigma-Aldrich | W384704-100G | MKBQ7148V |
| 20 | Maleic acid (sodium salt) | Sigma-Aldrich | M5757-25G | 089H5418V |
| 21 | Malic acid (sodium salt) | Sigma-Aldrich | M1125-25G | BCBF7491V |
| 22 | Methionine | Fluka | 64319 | 447626/1 |
| 23 | Proline | Sigma-Aldrich | P0380-100G | SLBJ6825V |
| 24 | Urea | Promega | V3175 | 174461 |
| 27 | DTT | Thermo-Scientific | 20291 | QH220094 |
| 28 | EDTA | Sigma-Aldrich | 9884-100G | SLBG0490V |
| 29 | Magnesium chloride | Sigma-Aldrich | M2393-100g | 019K00381V |
| 30 | Benzalkonium chloride | Sigma-Aldrich | 12063 | BCBl3282V |
| 31 | Brij 35 | Sigma-Aldrich | B4184-1L | 125K6039 |
| 32 | Poloxamer 188 (Pluronic F-68) | Spectrum | P1169 | UC0811 |
| 33 | Polysorbate 20 | Thermo-Scientific | 28320 | QE218997 |
| 34 | Polysorbate 80 | Thermo-Scientific | 28328 | QC217299 |
| 35 | Sodium docusate | Sigma-Aldrich | D1685-100G | SLBD7991V |
| 36 | Triton X-100 | Thermo-Scientific | 28314 | NA166964 |
| 37 | Lactose | Fluka | 17814-1KG | BCBK3531V |
| 38 | Sucrose | Pfanstiehl | S-124-1 | 35571A |
| 39 | Trehalose | Pfanstiehl | T-104-4 | 35261A |
| 40 | Glycerol | Sigma-Aldrich | G7893-2L | SHBF9304V |
| 41 | Mannitol | Pfanstiehl | M-109-6 | 35337A |
| 42 | Sorbitol | Sigma-Aldrich | S7547-1KG | SLBG0101V |
| 43 | Gamma-Cyclodextrin | Sigma-Aldrich | C4892-5G | SLBB4943V |
| 44 | 2-OH propyl b-CD | Sigma-Aldrich | H107-100G | SLBF6585V |
| 45 | Sulfobutyl ether beta-cyclodextrin | Captisol | RC-0C7-K01 | NC-04A-05033 |
| 46 | Carboxymethyl cellulose | Sigma-Aldrich | C9481-500G | SLBF4100V |
| 47 | Dextran sulfate | Spectrum | DE136 | TK1409 |
| 48 | Dextran 40 | Sigma-Aldrich | 31389-25G | BCBK7714V |
| 49 | PEG-3350 | Spectrum | P0125 | 2DH0463 |
| 50 | Sodium Hyaluronate | Acros | 251770250 | A0350849 |
| 51 | Calcium heptagluconate | Sigma-Aldrich | 21160-250G-F | 13228992 |
| 52 | Heparin | Sigma-Aldrich | H3393-500KU | 125K1336 |
| 53 | Maltose | Sigma-Aldrich | PHR1497-1G | LRAA304 |
| 54 | Vaxxas base formulation | Vaxxas | | |

Dried on disc samples in TPP plates were sealed with thermo-stable film and stored with desiccant at 48° C. for 7, 14, or 28 days. Samples were prepared on different days so that all samples could be collected and analyzed on the same day. The sample recovery method utilized 200 μL DPBS which was added to each well with disc and the plate was sealed with adhesive film. The plate was shaken at room temperature for 30 min at 200 rpm, and then the plate was sonicated in an ice water bath (the top of the plate was covered with a damp ice cold KimWipe) for 30 sec, 3 times, with 1 min intervals on ice. The plate was then centrifuged to collect condensation and each well was then manually mixed 15 times using electronic multichannel pipette (speed 5/9 and 100 μL/mix).

EIA assay was prepared as follows. EIA plate preparation was performed by taking Nunc Maxisorp 96 well plates and coating with 100 μL/well of anti-A/Cal mAb (1:4000 diluted in 0.1M sodium bicarbonate). The plates were wrapped with plastic wrap and aluminum foil, and incubated at 4° C. overnight. The following day, the plates were washed once with 200 μL PBST/well and blocked with 200 μL of 4 mg/mL BSA in PBS at room temperature for 1 hr. The plates were then stored with blocking solution at −20° C. until use. EIA plates and assay reagents (4 mg/mL BSA in PBS and PBST solutions) were thawed at room temperature. In a deep-well plate, 8 μg/mL HA standard was serial diluted with 4 mg/mL BSA in PBS to a final concentration of 4, 2, 1, 0.5, 0.25, 0.13, 0.063, 0.031, 0.016, and 0.0078 μg/mL HA. Ten μL of recovered MPH extract (by reverse pipetting) from each experimental well was diluted 1:45, 1:90, or 1:120 with 4 mg/mL BSA in PBS and manually mixed five times (300 μL/mix). The blocking solution from the EIA plate was then discarded and 100 μL of the HA standards or experimental MPH diluents were transferred to corresponding wells in the EIA plate. After incubation for 2-2.5 hrs at room-temperature, the plate was washed three times with PBST. One hundred μL of diluted Mab-HRP (stock Mab-HRP was diluted 1:3000 in 4 mg/mL BSA in PBS) was added to each well and incubated at room-temperature for 1.5 hrs. The plate was washed four times with PBST and then 70 μL of TMB substrate was added to each well for 11 min (plate was kept in dark during incubation). Then 70 μL of 1M HCL was added to each well to stop the reaction. The plate was read immediately at 450 nm (Molecular Devices, Spectra Max M5 microplate readers). ProMax software was used for data analysis.

The BCA assay was performed as follows. Fifty μL of BSA standards (0, 50, 100, 150, 200, 300, and 400 μg/mL BSA diluted in WFI), or recovered dried-on disc MPH samples (by reverse pipetting), were transferred to corresponding wells in a TPP plate. Two hundred μL of the BCA reagent (diluted 1:50 with WFI) was added to each well. The plates were incubated at 37° C. for 40 min, and absorbance was measure at 562 nm (Molecular Devices, Spectra Max M5 microplate readers). ProMax software was used for data analysis.

Viscosity measurement: 250 μL of MPH was mixed with equal volume of excipient or DPBS solution (formulated MPH contains 3.0 mg/mL HA). The viscosity of each condition was measured (in triplicate) using a m-VROC viscometer, at a flow rate of 100 μL/min, for 20 second, at 25° C.

MPH was mixed with equal volume of 2× stock excipients (Table 2.1) or DPBS. Five μL of each MPH solution was then dispensed onto the center of each disc (15 μg HA/disc) and dried under N₂ flow. In total, fifty-three different excipients were tested. The Vaxxas base formulation (0.6% (w/w) hypromellose and 0.4% (w/w) trehalose dehydrate in DPBS) and a DPBS-alone (no excipient) formulation were also included as controls for relative comparisons. All samples (in quadruplet) were then incubated for 7 days at 48° C. Corresponding formulations alone (without MPH) and the MPH in DPBS-alone (control) was included in each plate. After the incubation, sample was recovered using method #3 and the protein recovery and HA potency of each sample was analyzed using BCA and EIA assays, respectively. Please note that all values (recovery and potency) were normalized to the Day 0 sonicated MPH solution control sample and all values have had their respective excipient alone values subtracted.

For the BCA assay, 12 formulations (#26, 45, 25, 44, 12, 43, 37, 38, 14, 13, 53, and 21) achieved >80% HA protein recovery, and 5 formulations (#48, 16, 54, 39, and 19) achieved 60-80% HA protein recovery. Some reducing sugars and protein excipients alone interfered with BCA assay. For the EIA assay, 7 formulations (#38, 13, 37, 12, 45, 26, and 43) achieved >80% HA potency recovery, and 12 formulations (#9, 44, 25, 14, 39, 16, 32, 21, 19, 54, 53, and 48) achieved 60-80% HA potency recovery. Normalized HA potency rates (i.e., EIA/BCA) are not reported because low protein recovery and low HA potency values would have similar EIA/BCA values compared to high recovery and potency values, and therefore the normalized potency rate was not an appropriate metric to identify stabilizing excipients.

Formulation additives that achieved >80% in both protein recovery and HA potency are summarized in Table 2. These additives consisted of two sugars (sucrose and lactose), two individual amino acids (arginine and aspartic acid), an amino acid mixture (arginine, glutamic acid, and isoleucine), and two cyclodextrins (Sulfobutyl ether beta-cyclodextrin and gamma-cyclodextrin). Conversely, glycerol had the strongest negative effect (i.e., low recovery and potency) compared to all other excipient. Additionally detergents (e.g., Triton X-100) appeared to interfere with the formation of the MPH droplet on the disk and after drying, MPH flakes were observed, which were prone to detach from the discs.

TABLE 2
Excipients that achieved >80% HA protein recovery (BCA
assay) and HA potency (EIA assay) after storage for 7
days at 48° C. in a dried state in a DPBS base buffer.

| Excipient | | EIA potency | BCA recovery |
|---|---|---|---|
| category | HA + Excipient | rate % | rate % |
| Carbohydrates | 2% Sucrose | 93 ± 18% | 92 ± 10% |
| | 2% Lactose | 90 ± 13% | 92 ± 7% |
| Amino Acids | 0.1M Aspartic acid | 93 ± 5% | 89 ± 6% |
| | 0.1M Arginine | 88 ± 8% | 92 ± 4% |
| | 0.045M Arginine + | 87 ± 12% | 98 ± 5% |
| | 0.045M Glutamic acid + | | |
| | 0.01M Isoleucine | | |
| Cyclodextrins | 5% Sulfobutyl ether | 87 ± 14% | 96 ± 6% |
| | beta-cyclodextrin | | |
| | 5% Gamma-cyclodextrin | 81 ± 8% | 92 ± 7% |

### Example 2

### Stability Testing of Individual Excipients

Five excipients from different categories (2% sucrose, 0.1M Apartic acid, 0.1M Arginine, an amino acid mixture (0.045M Arginine+0.045M Glutamic acid+0.01M Isoleucine), and 5% Sulfobutyl ether beta-cyclodextrin) were chosen for further testing to identify candidate MPH formulations. As shown in Table 3, three concentrations of each lead excipient were incubated with MPH at 48° C. for 0, 7, 14, and 28 days. Please note that unlike the initial excipient screen, the incubation duration was extended to 28 days in this study in an attempt to better differentiate the stabilizing effects of each excipient concentration.

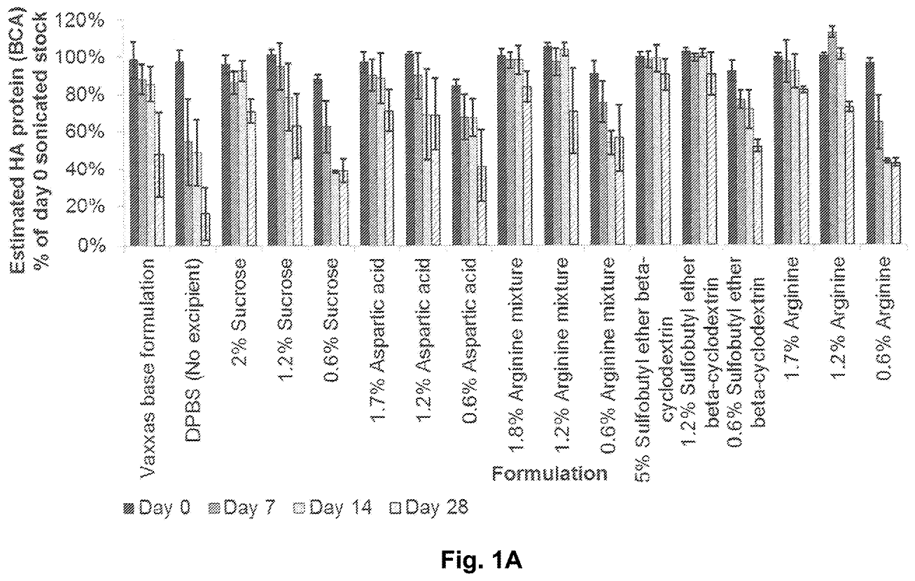

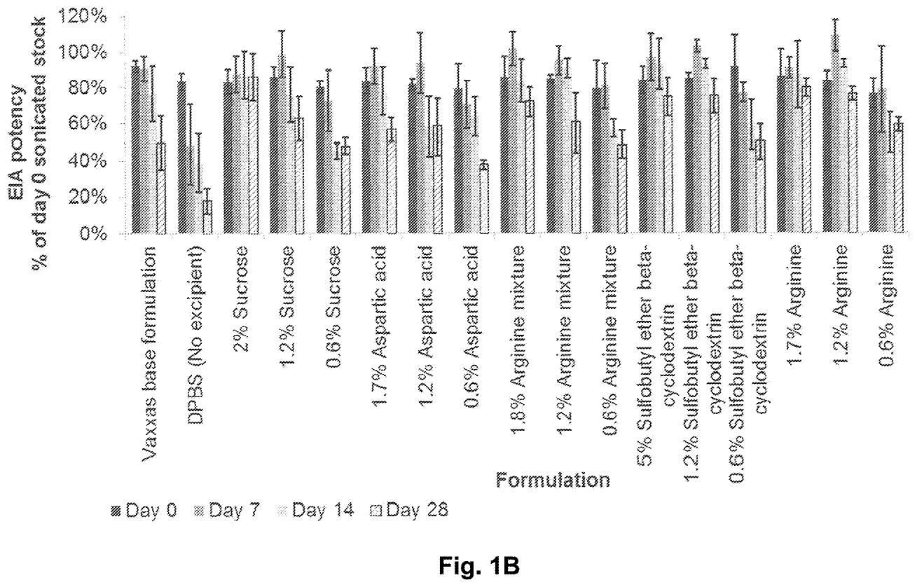

As shown in FIGS. 1A and 1B, the relative protein recovery and HA potency decreased by varying extents over time, but most of the tested formulations achieved >50% recovery and potency after 28 days incubation at 48° C. By Day 28, the protein recovery and HA potency trended higher with higher excipients concentrations; however, the maximal concentration of each lead excipient exceeded the 1.2% (w/v) weight limit designated (Table 4).

TABLE 3
Excipient concentration screening study.
A

| Excipient | | |
|---|---|---|
| category | HA + Excipient | Concentrations to test (C1, C2, and C3) |
| Carbohydrates | Sucrose | 2% (w/v), 1.2% (w/v), and 0.6% (w/v) |
| Amino Acids | Aspartic acid | 0.1M: equivalent to 1.7% (w/v), |
| | | 0.07M: equivalent to 1.2% (w/v), |
| | | 0.035M: equivalent to 0.6% (w/v) |
| | Arginine + | 45 mM, 45 mM, 10 mM: equivalent to 1.8% (w/v), |
| | Glutamic acid + | 30 mM, 30 mM, 6.7 mM: equivalent to 1.2% (w/v), |
| | Isoleucine | 15 mM, 15 mM, 3.4 mM: equivalent to 0.6% (w/v) |
| | Arginine | 0.1M: equivalent to 1.7% (w/v), |
| | | 0.07M: equivalent to 1.2% (w/v), |
| | | 0.03M: equivalent to 0.6% (w/v) |
| Cyclodextrins | Sulfobutyl ether | 45 mM, 45 mM, 10 mM: equivalent to 1.8% (w/v), |
| | beta-cyclodextrin | 30 mM, 30 mM, 6.7 mM: equivalent to 1.2% (w/v), |
| | | 15 mM, 15 mM, 3.4 mM: equivalent to 0.6% (w/v) |

B

| | 1 | 2 | 3 | 4 | 5 | 6 | 7 | 8 | 9 | 10 | 11 | 12 |
|---|---|---|---|---|---|---|---|---|---|---|---|---|
| A | Left blank | | | | | | | | | | | |
| B | excipient | | excipient | | excipient | | excipient | | Sonicated stock | | | |
| | E1C1 | | E2C1 | | E3C1 | | E4C1 | | | | | |
| C | HA + excipient E1C1 | | | | HA + excipient E1C2 | | | | HA + excipient E1C3 | | | |
| D | HA + excipient E2C1 | | | | HA + excipient E2C2 | | | | HA + excipient E2C3 | | | |
| E | HA + excipient E3C1 | | | | HA + excipient E3C2 | | | | HA + excipient E3C3 | | | |
| F | HA + excipient E4C1 | | | | HA + excipient E4C2 | | | | HA + excipient E4C3 | | | |
| G | HA + Vaxxas | | | | HA + no excipient (DPBS) | | | | Stock | | | |
| H | Left blank | | | | | | | | | | | |
| | 1 | 2 | 3 | 4 | 5 | 6 | 7 | 8 | 9 | 10 | 11 | 12 |

| A | Left blank | | |
|---|---|---|---|
| B | | | |
| C | excipient | | Sonicated stock |
| | E5C1 | | |
| D | HA + excipient E5C1 | HA + excipient E5C2 | HA + excipient E5C3 |
| E | HA + Vaxxas | HA + no excipient (DPBS) | Stock |
| F | Left blank | | |
| G | | | |
| H | | | |

(A) Three concentrations of each lead excipients were screened. C1 was the original concentration tested in section 2.3.1, C2 was the maximal weight limit (1.2% w/v) of each lead excipient, and C3 was a half-maximal weight limit (0.6% w/v) of each lead excipient.
(B) Representative TPP plate layout of discs (BCA and EIA assays followed the same plate layout.).

TABLE 4
Protein recovery and HA potency of MPH samples after storage for
28 days at 48° C. in DPBS base buffer in a dried state formulated
with three different concentrations of each excipient (in quadruplicate).
Excipient concentrations exceeding the 1.2% (w/v) maximal weight
limit designated by Vaxxas are listed in blue.

| HA + Excipient | BCA Recovery | EIA Potency |
|---|---|---|
| Day 0 sonicated stock | 100 ± 2% | 100 ± 12% |
| 1.2% Sulfobutyl ether beta-cyclodextrin | 91 ± 11% | 75 ± 10% |
| 5% Sulfobutyl ether beta-cyclodextrin | 90 ± 8% | 75 ± 11% |
| 1.8% Arginine mixture | 84 ± 8% | 72 ± 8% |
| 1.7% Arginine | 82 ± 2% | 79 ± 5% |
| 1.2% Arginine | 73 ± 3% | 76 ± 4% |
| 1.7% Aspartic acid | 72 ± 11% | 57 ± 7% |
| 2% Sucrose | 71 ± 7% | 86 ± 13% |
| 1.2% Arginine mixture | 71 ± 23% | 61 ± 16% |
| 1.2% Aspartic acid | 69 ± 19% | 58 ± 15% |
| 1.2% Sucrose | 63 ± 17% | 63 ± 12% |
| 0.6% Arginine mixture | 56 ± 18% | 49 ± 8% |
| 0.6% Sulfobutyl ether beta-cyclodextrin | 52 ± 3% | 50 ± 10% |
| Vaxxas base formulation | 48 ± 23% | 50 ± 15% |
| 0.6% Arginine | 43 ± 2% | 59 ± 4% |
| 0.6% Aspartic acid | 42 ± 19% | 38 ± 3% |
| 0.6% Sucrose | 39 ± 6% | 48 ± 4% |
| DPBS (No excipient) | 16 ± 14% | 18 ± 7% |

### Example 3

### Stability Testing of Combinations of Excipients

To determine if combinations of excipients would further improve the protein and HA potency recovery, three of the lead excipients from different categories (sucrose, arginine, and sulfobutyl ether beta-cyclodextrin) were chosen for further combination analysis. These excipients were tested in combination (Excipient #1+Excipient #2) at three different ratios comprising the 1.2% (w/v) weight limit (Table 2.5), or a combination containing all three excipients (0.4% (w/v) each, 1.2% (w/v) total). In consultation with Vaxxas, possible synergistic effects of excipients was also tested that consisted of a combination of all three excipients at 1.2% (w/v) each, and combinations of two excipients at 1.2% (w/v) each. Controls included each lead excipient at 1.2% (w/v), Vaxxas base formulation, and DPBS alone (no excipient). All formulations were prepared on the same plate to minimize plate-to-plate recovery variation.

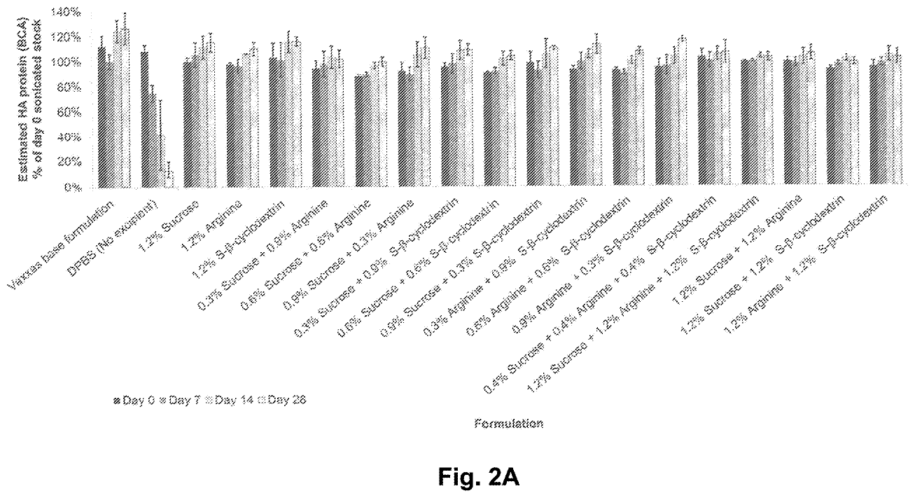

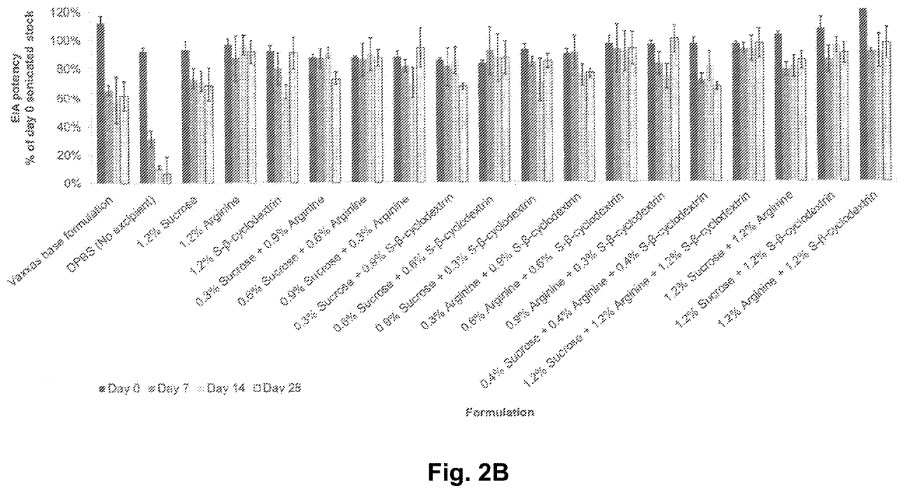

As shown in FIG. 2A, all formulations (individual excipients or in-combination) achieved >98% protein recovery except for the DPBS-alone (no excipient) condition. Furthermore, all of the tested excipient combinations achieved >65% HA potency after 28 days of incubation at 48° C. The combination containing all three excipients (0.4% (w/v) each) achieved the lowest HA potency among all tested individual and in-combination conditions. Additionally, there was no apparent synergistic effect observed in excipient combinations exceeding the 1.2% (w/v) weight limit (Table 5).

TABLE 5
Protein recovery and HA potency of MPH samples after storage for
28 days at 48° C. in a DPBS base buffer in a dried state formulated
with various combinations of lead excipients (in quadruplicate).

| | | EIA | BCA |
|---|---|---|---|
| | Formulation | Potency | Recovery |
| Excipients | 0.9% Arginine + 0.3% S-β-cyclodextrin | 100 ± 10% | 116 ± 2% |
| (individual or | 0.9% Sucrose + 0.3% Arginine | 94 ± 14% | 110 ± 9% |
| combination) within | 0.6% Arginine + 0.6% S-β-cyclodextrin | 93 ± 12% | 107 ± 3% |
| the 1.2% weight limit | 1.2% Arginine | 91 ± 8% | 110 ± 5% |
| | 1.2% S-β-cyclodextrin | 90 ± 11% | 116 ± 4% |
| | 0.6% Sucrose + 0.6% Arginine | 87 ± 6% | 99 ± 4% |
| | 0.6% Sucrose + 0.6% S-β-cyclodextrin | 87 ± 12% | 104 ± 4% |
| | 0.9% Sucrose + 0.3% S-β-cyclodextrin | 84 ± 5% | 110 ± 2% |
| | 0.3% Arginine + 0.9% S-β-cyclodextrin | 76 ± 2% | 113 ± 8% |
| | 0.3% Sucrose + 0.9% Arginine | 74 ± 4% | 102 ± 7% |
| | 1.2% Sucrose | 69 ± 11% | 115 ± 7% |
| | 0.3% Sucrose + 0.9% S-β-cyclodextrin | 68 ± 2% | 108 ± 4% |
| | 0.4% Sucrose + 0.4% Arginine + 0.4% | 68 ± 3% | 106 ± 9% |
| | S-β-cyclodextrin | | |
| Excipients | 1.2% Sucrose + 1.2% Arginine + 1.2% | 97 ± 10% | 102 ± 3% |
| (combination) | S-β-cyclodextrin | | |
| exceeding the | 1.2% Arginine + 1.2% S-β-cyclodextrin | 96 ± 11% | 102 ± 6% |
| 1.2% weight limit | 1.2% Sucrose + 1.2% S-β-cyclodextrin | 89 ± 7% | 98 ± 3% |
| | 1.2% Sucrose + 1.2% Arginine | 84 ± 6% | 105 ± 6% |
| Control samples | Sonicated stock Day 0 | 100 ± 4% | 100 ± 3% |
| | Base formulation | 62 ± 11% | 126 ± 12% |
| | DPBS (No excipient) | 6 ± 12% | 14 ± 5% |

Fifty-four excipients (including the base formulation) were screened to increase the recovery and potency of HA in a dried state. After 7 days of incubation at 48° C., 7 excipients (2 carbohydrates, 2 amino acids, an amino acid combination, and 2 cyclodextrins) achieved >80% protein and HA potency recovery (BCA and EIA assays, respectively). Five out of these 7 lead excipients were selected for additional concentration optimization to meet the weight limits of the Inkjet process (1.2% (w/v)). After 28 days of incubation at 48° C., three excipients (sucrose, arginine, and sulfobutyl ether beta-cyclodextrin) achieved >60% protein and HA potency recovery at concentrations of 1.2% (w/v) or lower. These three excipients were further screened in various combinations, which achieved >65% HA potency and >98% protein recovery after 28 days storage at 48° C. Finally, all of the candidate formulations with indicated levels of excipients in DPBS solution (Table 6) have viscosity values ranging from 1.3-2.3 cP.

TABLE 6
Stabilizing additives for A/California/Jul. 2009 MPH in a dried state.
Formulations (Excipient(s) + DPBS)

1.2% Sucrose
1.2% Arginine
1.2% Sulfobutyl ether β-cyclodextrin
0.3% Sucrose + 0.9% Arginine
0.6% Sucrose + 0.6% Arginine
0.9% Sucrose + 0.3% Arginine
0.3% Sucrose + 0.9% Sulfobutyl ether β-cyclodextrin
0.6% Sucrose + 0.6% Sulfobutyl ether β-cyclodextrin
0.9% Sucrose + 0.3% Sulfobutyl ether β-cyclodextrin
0.3% Arginine + 0.9% Sulfobutyl ether β-cyclodextrin
0.6% Arginine + 0.6% Sulfobutyl ether β-cyclodextrin
0.9% Arginine + 0.3% Sulfobutyl ether β-cyclodextrin
0.4% Sucrose + 0.4% Arginine + 0.4% Sulfobutyl ether β-cyclodextrin
1.2% Sucrose + 1.2% Arginine + 1.2% Sulfobutyl ether β-cyclodextrin
1.2% Sucrose + 1.2% Arginine
1.2% Sucrose + 1.2% Sulfobutyl ether β-cyclodextrin
1.2% Arginine + 1.2% Sulfobutyl ether β-cyclodextrin

Testing of HA from A/California on microprojection array with Sulpho-Butyl Ether Beta-Cyclodextrin (SBECD) in medican with desiccant provided:
1 month 2-8 C—no change in HA potency;
1 month 48 C—20% reduction in HA potency;
3 month 2-8 C—no change in HA potency; and
3 month 25 C—22% reduction HA in potency (TBC, issue with sonication of material).

Testing of HA from A/California on microprojection array with L-arginine In medican with desiccant:
3 month 2-8 C—no change in HA potency;
3 month 25 C—no change in HA potency; and
Excellent recovery off the patch at 3 months.

Testing of HA from A/South Australia on microprojection array with Sulpho-Butyl Ether Beta-Cyclodextrin in medican with desiccant:
1 month 2-8 C—no change in HA potency; and
1 month 48 C—no change in HA potency.

Testing of HA from B/Phuket Australia on microprojection array with Sulpho-Butyl Ether Beta-Cyclodextrin in medican with desiccant:
Approx. 35% drop in potency on dry down.

### Example 5

Stability Testing of Excipients for Trivalent Inactivated Polio Vaccine (tIPV)

Approximately 15 mL of Inactivated poliomyelitis vaccine type 1 (IPV1) (Batch PV11—158B, containing 1250-3140 DU/mL D-antigen, manufactured by Bilthoven Biologicals, Cyrus Poonawalla Group), 10 mL of Inactivated poliomyelitis vaccine type 2 (IPV2) (Batch PV09-224B, containing 430-1480 DU/mL D-antigen, manufactured by Bilthoven Biologicals, Cyrus Poonawalla Group), and 25 mL of Inactivated poliomyelitis vaccine type 3 (IPV3) (Batch PV09—335B, containing 520-2220 DU/mL D-antigen, manufactured by Bilthoven Biologicals, Cyrus Poonawalla Group), were provided. These three monovalent IPV stock solutions were stored at 4° C. and were used.

Concentrated (4×) stock solutions of excipients were prepared by dissolving compounds in DPBS (pH 7.2), adjusting the pH to 7.2 using HCl or NaOH, and sterilizing the solutions by filtering through 0.22 μm PVDF membrane (the first 5 mL of each solution passing through the PVDF membrane was discarded to eliminate any potential contamination of residual particles or extractables from the filters). The excipient stock solutions were stored at 4° C. or room temperature (if the solution precipitated at 4° C.) for up to 2 weeks (unstable excipients such as reducing agents were prepared immediately before use). Excipients tested included Histidine, Sodium acetate, Sodium chloride, Sodium citrate, Sodium phosphate, Sodium sulfate, Sodium succinate, Gelatin, Hydrolysed Gelatin, Protamine sulfate, Arginine, Aspartic acid (sodium salt), Glutamic acid, Glycine, Isoleucine, Lactic acid, Lysine, Maleic acid, Malic acid (sodium salt), Methionine, Urea, EDTA, Magnesium chloride, Benzalkonium chloride, Brij 35, Poloxamer 188 (Pluronic F-68), Polysorbate 20, Polysorbate 80, Sodium docusate, Triton X-100, Lactose, Sucrose, Trehalose, Glycerol, Mannitol, Sorbitol, Gamma-Cyclodextrin, 2-OH propyl b-CD, Sulfobutyl ether beta-cyclodextrin, Carboxymethyl cellulose, Dextran sulfate, Dextran 40, PEG-3350, Sodium Hyaluronate, Sodium thioglycolate, Cysteine, and Glutathione.

LCP (liquid crystal polymer) discs were placed into TPP® 96 well plates (1 disc/well). There monovalent IPV bulk solutions were mixed to make a tIPV solution containing 40 parts of IPV1, 8 parts of IPV2, and 32 parts of IPV3 (in D-antigen units). 7.5 of tIPV mixture in M199 media was further diluted with 2.5 μL of 4× excipient or DPBS (The final formulation is in % M199 and ¼ DPBS. This buffer is referred as “M199/DPBS” in the text). 10 μL of formulated tIPV was dispensed onto the center of each disc (equivalent to 1/9-⅙ of full human dose/disc). Please note that the values of the IPV bulk solutions varied depending on the vial of IPV standard used to calculate the D-antigen concentration in each bulk solution. The plate was then transferred to the drying rig, dried for 17-19 min under 14 L/min N2 flow, and then sealed with thermo-stable adhesive film.

Dried on disc samples in TPP® plates were sealed with thermo-stable film and stored with a bag of desiccant (anhydrous calcium sulfate, from Drierite) at indicated temperature and period of time. Samples were prepared on the same day and assayed on the different days. For recovery during the assay, 200 μL reconstitution buffer (DPBS with 1% of BSA and 0.1% PS80, pH 7.2, and filtered through 0.22 μm PVDF filter) was added to each well with disc and the plate was sealed with adhesive film. The plate was shaken at room temperature for 30 min at 200 rpm. Each well was then manually mixed ten times using electronic multichannel pipette (speed 5/9 and 100 μL/mix). The PS80 and BSA concentrations in the reconstitution buffer were increased to 0.5% and 2%, respectively, to potentially improve the recovery of samples stored for longer durations at higher temperatures.

To evaluate IPV vaccine stability following drying, D-antigen values of recovered vaccine were determined using the ELISA assay described in Example 1. The percent potency of recovered dried vaccine was calculated by normalizing the D-antigen values of recovered dried samples to the values of an in-liquid stock vaccine stored at 4° C., which was considered to have 100% potency. The drying potency loss was calculated by subtracting the percent potency of freshly dried vaccine samples (recovered immediately after drying) from the in-liquid stock vaccine stored at 4° C. (i.e., 100%−relative percent potency after drying=drying potency loss). Similarly, the storage potency loss was determined by subtracting the relative potency of the stored samples with the relative percent potency of the sample recovered immediately after drying (i.e., 100%−relative percent potency after storage−relative percent potency after drying=storage potency loss). Errors for losses were calculated by propagation of error method using following equation SE(C)=√(SE(A)²+SE(B)²).

A reconstitution solution consisting of DPBS buffer alone was only able to recover a small portion of the on-disc tIPV samples (freshly dried or stability) during the D-antigen potency assay. A new reconstitution buffer was needed to improve sample recovery. After screening combinations of 0-1% PS80 and 0-5% BSA, a new reconstitution solution (DPBS buffer containing 0.1% PS80 and 1% BSA, pH 7.2) was found to greatly improve the D-antigen potency of tIPV samples dried on the discs. Table 7 summarizes the potency of freshly dried tIPV sample, dried and stored for 1 day at 4° C., or dried and stored for 7 days at 4° C.

TABLE 7
Potency of tIPV sample for different drying conditions.

| | IPV 1 | IPV 2 | IPV 2 |
|---|---|---|---|
| Sample | Potency | Potency | Potency |
| (Reconstituted Buffer) | Loss (%) | Loss (%) | Loss (%) |
| Freshly dried (DPBS) | 52 ± 2% | 54 ± 3% | 83 ± 2% |
| Freshly dried | 12 ± 6% | 0 ± 4% | 29 ± 7% |
| (DPBS + 0.1% PS80 + 1% BSA) | | | |
| Dried and Stored for 1 Day 4° C. | 78 ± 0% | 70 ± 1% | 93 ± 0% |
| (DPBS) | | | |
| Dried and Stored for 1 Day 4° C. | 48 ± 4% | 10 ± 3% | 82 ± 1% |
| (DPBS + 0.1% PS80 + 1% BSA) | | | |
| Dried and Stored for 7 Day 4° C. | 82 ± 1% | 71 ± 2% | 92 ± 1% |
| (DPBS) | | | |
| Dried and Stored for 7 Day 4° C. | 59 ± 1% | 25 ± 3% | 84 ± 1% |
| (DPBS + 0.1% PS80 + 1% BSA) | | | |

Over 30 and 50% D-antigen potency losses were observed for IPV3 in M199/DPBS immediately after drying and after 7 days storage at 4° C., respectively (using the optimized reconstitution buffer of DPBS with PS80 and BSA). The substantial loss of potency (˜80% total potency loss) from these conditions provided a stability indicating assay to screen for stabilizing excipients. In the initial excipient screen, tIPV was mixed with one third volume of 4× stock excipients in DPBS buffer (Table 2.1) or DPBS alone (control). Ten μL of each formulated tIPV solution was then dispensed onto the center of each disc (equivalent to 1/9-⅙ of full human dose/disc) and dried under N₂ flow. In total, fifty-one different excipients were tested in M199/DPBS. All samples (in quadruplet) were then recovered immediately after drying, or after incubation for 7 days at 4° C. for D-antigen ELISA analysis. Corresponding formulations alone (without tIPV) and the tIPV in DPBS-alone (control) were also included in each plate No interference/background signal was detected from the excipients alone samples in the ELISA assay, and all potency percent loss values were obtained by normalizing results to the Day 0 tIPV stock solution control sample. Drying potency loss was calculated by subtracting the percent potency of freshly dried vaccine samples (recovered immediately after drying) from the in-liquid stock vaccine stored at 4° C. (i.e. 100%−relative percent potency after drying=drying potency loss). Similarly, storage potency loss was determined by subtracting the relative potency of the stored samples with the relative percent potency of the sample recovered immediately after drying (i.e. 100%−relative percent potency after storage−relative percent potency after drying=storage potency loss).

Potency loss for each IPV serotype during dying and storage for 7 days at 4 C indicated that some excipients mitigated potency loss while other excipients appeared to exacerbate potency loss in each IPV serotype. Trends between excipient categories were also observed. For example in IPV2, potency loss during drying was higher when amino acids were present in the buffer compared to the control but potency loss during drying in other excipient categories (e.g. carbohydrates, polyols) were lower than the control sample. For the least stable serotype (IPV3), potency loss in 29 formulations immediately after drying and recovery were lower than the M199/DPBS alone control, and 34 excipients mitigated IPV3 potency loss during storage for 7 days at 4° C. better than the control sample. Excipients providing improved stability from the initial screening are summarized in Table 8, which consisted of a reducing agent (DTT), two individual amino acids (Arginine, Histidine), an amino acid mixture (Arginine, Glutamic acid, with or without Isoleucine), two carbohydrates (Sucrose and Lactose), three cyclodextrins (γ-Cyclodextrin, 2-OH propyl β-Cyclodextrin, and SBE-(3-Cyclodextrin), salt/buffer Tris, and from one additive from the protein category (gelatin). Conversely, detergents (e.g., Triton X-100) appeared to interfere with the formation of the tIPV droplet on the disc and after drying, since tIPV flakes were observed after drying.

TABLE 8
Excipients that provided superior stability.

| | | Total Potency Loss | | |
|---|---|---|---|---|
| | | (after drying and 7 | | |
| Excipient | | days storage at 4° C.) | | |
| category | Excipient | IPV 1 | IPV2 | IPV3 |
| Reducing agent | 1 mM DTT (0.016% (w/v)) | 36 ± 1% | 17 ± 1% | 32 ± 2% |
| Amino Acids | 0.1M Arginine (1.7% (w/v)) | 23 ± 3% | 9 ± 2% | 36 ± 4% |
| | 45 mM Arg + 45 mM Glu + | 30 ± 5% | 16 ± 2% | 48 ± 8% |
| | 10 mM Ile (1.8% (w/v)) | | | |
| | 50 mM Arg + 50 mM Glu | 32 ± 2% | 16 ± 1% | 53 ± 2% |
| | (1.8% (w/v)) | | | |
| | 60 mM Histidine (1.8% (w/v)) | 38 ± 2% | 7 ± 2% | 58 ± 2% |
| Carbohydrates | 1.6% (w/v) sucrose | 26 ± 3% | 20 ± 1% | 62 ± 2% |
| | 2% (w/v) lactose | 24 ± 5% | 14 ± 1% | 65 ± 3% |
| Cyclodextrins | 5% (w/v) Υ-cyclodextrin | 38 ± 3% | 17 ± 1% | 63 ± 4% |
| | 5% (w/v) 2-OH propyl | 42 ± 3% | 14 ± 2% | 67 ± 5% |
| | β-cyclodextrin | | | |
| | 5% (w/v) SBE-β-cyclodextrin | 43 ± 8% | 21 ± 10% | 68 ± 4% |
| Salts/Buffers | 50 mM Tris (TromeThamine) | 38 ± 4% | 22 ± 2% | 67 ± 2% |
| | 0.6% w/v | | | |
| Protein | 1% (w/v) gelatin | 44 ± 1% | 21 ± 3% | 68 ± 2% |

Each of the excipients from Table 8 was chosen for further concentration review to identify candidate tIPV formulations. In this second excipient screening study, the lead excipient DTT was substituted for reducing agents listed on the FDA inactive ingredient guide, including: Sodium thioglycolate, Cysteine, and Glutathione. These reducing agents were each screened at 20 mM, 5 mM, and 1 mM with tIPV using the same conditions as the initial excipient screening study. All other excipients listed in Table 8 were further tested at multiple concentrations (2×, 1×, and 0.5×) with tIPV using the same conditions as the initial excipient screening study (Please note: Sucrose, Lactose, and Histidine could not exceed 1× due to insufficient drying or solubility issues. Therefore, only 1× and 0.5× of these excipients were tested).

Sodium thioglycolate, Cysteine, and Glutathione showed similar or a better stabilizing effect during storage with each IPV serotype compared to DTT. For instance, potency loss of IPV3 was minimal (<5%) in 5 or 20 mM Glutathione or Cysteine after storage for 7 days at 4° C., compared to −35% for 1 mM DTT. Overall, 20 mM Glutathione was observed to be the best reducing agent excipient in which the potency loss of IPV3 was −20% after drying and storage for 7 days at 4° C., compared to −96% in the DPBS alone control sample. While these reducing agents mitigated potency loss during storage, their stabilizing effect for potency loss during drying was minimal. When screening the effect of the other lead excipients (other than reducing agents) on tIPV stability, many improved (lowered) potency losses during drying or during storage at 4° C. for 7 days, but generally not both. For example, the cyclodextrins mitigated potency loss during drying but did not appear as beneficial during storage, while the carbohydrates or amino acids were not as useful during drying but mitigated potency loss during storage. A summary of best stabilizing excipients for drying of tIPV and best additives for storage of tIPV in the dried state are provided in Tables 9 and 10.

TABLE 9
Potency loss of IPV1, IPV2 and IPV3 during drying.

| Excipient | | Potency Loss During Drying | | |
|---|---|---|---|---|
| category | Excipient | IPV 1 | IPV2 | IPV3 |
| Reducing agent | 20 mM glutathione | 5% | −2% | 21% |
| | 1 mM cysteine | 9% | −3% | 27% |
| Cyclodextrins | 5% (w/v) SBE-β- | 5% | 0% | 8% |
| | cyclodextrin | | | |
| | 5% (w/v) 2-OH propyl | 5% | −1% | 10% |
| | β-cyclodextrin | | | |
| | 5% (w/v) Υ-cyclodextrin | 3% | −2% | 15% |
| Protein | 1% gelatin | 4% | 1% | 10% |

TABLE 10
Potency loss of IPV1, IPV2 and IPV3 during storage.

| Excipient | | Potency Loss During Storage | | |
|---|---|---|---|---|
| category | Excipient | IPV 1 | IPV2 | IPV3 |
| Carbohydrates | 2% lactose | 20% | 12% | −19% |
| | 1.6% sucrose | 28% | 13% | 2% |
| Amino Acids | 30 mM Histidine | 26% | 11% | −8% |
| | 0.2M arginine | 10% | −26% | −4% |
| Reducing agents | 20 mM cysteine | 7% | −5% | −5% |
| | 120 mM glutathione | −12% | −10% | −3% |
| Cyclodextrins | 8.5% (w/v) | 5% | −27% | 10% |
| | Υ-cyclodextrin | | | |

Cyclodextrins or gelatin mitigated IPV potency loss during drying, while carbohydrates or amino acids mitigated potency loss during storage in the dried state. Combinations of excipients from these different categories were therefore tested to determine if tIPV potency loss can be further diminished during drying and storage. Due to the number of lead excipients for drying and storage, the study was divided into two steps. First, optimal combinations for drying were screened. Second, the optimized combinations giving maximal potency after drying were optimized with one or multiple lead excipients for storage in the dried state. In the first step, combinations of reducing agents (15 mM Glutathione and 1 mM Cysteine), and cyclodextrins (5% SBE-(3-CD and 2.5% γ-CD) were tested to mitigate potency loss during drying. In addition, 1% gelatin (type A gelatin and hydrolyzed gelatin) were tested individually or combined with 1 mM Cysteine and/or 5% SBE-β-CD. Controls included each lead excipient alone, and DPBS alone (no excipient). IPV3 potency losses in the presence of the different excipient combinations were all lower than in the DPB S control. Combinations of cyclodextrin+reducing agent appeared to mitigate potency loss the best during drying while potency loss was the highest in the reducing agents or gelatin alone samples. The optimal drying excipient combination included a cyclodextrin and a reducing agent; however, the type of cyclodextrin (5% SBE-β-Cyclodextrin or 2.5% γ-Cyclodextrin) and reducing agent (15 mM Glutathione or 20 mM Cysteine) combination could not be delineated from these results and was evaluated in subsequent studies.

In the second step, tIPV formulated with 5% SBE-β-Cyclodextrin or 2.5% γ-Cyclodextrin were tested in combinations with one or multiple stabilizing excipients for stability during storage in dried state. Potency losses were determined for tIPV immediately after drying and after storage for 7 days at 4° C. or 25° C. Formulations with cyclodextrins had the lowest IPV3 potency loss immediately after drying, which is consistent with the results in Step 1 (see above). While 20 mM Cysteine (alone or in combination with other excipients) mitigated potency loss for IPV3, this excipient appeared to destabilize (increase the potency loss) of IPV1. Cysteine, Glutathione, Histidine, and Arginine worked well in preventing potency loss for each of the three IPV serotypes during storage at 4° C. In samples stored at 25° C., Cysteine, Glutathione, Arginine, or their combination with Cyclodextrins worked well in minimizing potency loss for each of the three IPV serotypes. Overall, tIPV formulated with one of the cyclodextrins in combination with either Glutathione or Histidine achieved the lowest potency loss after drying and 7 days storage at 4° C. or 25° C. for IPV3.

From the excipient combination screen described above, two cyclodextrins (5% SBE-β-Cyclodextrin or 2.5% γ-Cyclodextrin) in combination with 15 mM Glutathione or 30 mM Histidine, resulted in the lowest total potency loss after drying and 7 days storage at 4° C. or for IPV3. In addition, 0.2 M Arginine and 20 mM Cysteine appeared to help prevent IPV potency loss during storage as well. In the next study, the performances of candidate tIPV formulations for longer storage periods were tested. Twenty-two formulations composed of one cyclodextrin (4.5% SBE-(β-Cyclodextrin or 2.5% γ-Cyclodextrin) for stabilization during drying, and 1-3 best excipients for stabilization during storage (15 mM Glutathione, 30 mM Histidine, 0.15 M Arginine, and/or 20 mM Cysteine), were tested for tIPV potency immediately after drying, and after 2, 3, 4 weeks storage at 4° C., and after 1, 2, 3 weeks storage at 25° C. (Please note that SBE-(β-Cyclodextrin concentration was decreased from 5% to 4.5%, and Arginine from 200 mM to 150 mM for proper drying). DPBS alone (no excipient) was included as a control. The tested formulations are listed in Table 11.

TABLE 11
List of excipient combinations tested.

| # | Excipient Combinations Tested |
|---|---|
| 1 | 4.5% SBE-beta-Cyclodextrin + 15 mM Glutathione |
| 2 | 2.5% γ-Cyclodextrin + 15 mM Glutathione |
| 3 | 2.5% γ-Cyclodextrin + 30 mM Histidine + 15 mM Glutathione |
| 4 | 4.5% SBE-beta-Cyclodextrin + 30 mM Histidine + 15 mM |
| | Glutathione |
| 5 | 4.5% SBE-beta-Cyclodextrin + 20 mM cysteine |
| 6 | 4.5% SBE-beta-Cyclodextrin + 150 mM Arginine + 15 mM |
| | Glutathione |
| 7 | 2.5% γ-Cyclodextrin + 30 mM Histidine |
| 8 | 4.5% SBE-beta-Cyclodextrin + 30 mM Histidine |
| 9 | 2.5% γ-Cyclodextrin + 20 mM Cysteine |
| 10 | 2.5% γ-Cyclodextrin + 15 mM Glutathione + 30 mM |
| | Histidine + 150 mM Arginine |
| 11 | 2.5% γ-Cyclodextrin + 150 mM Arginine + 15 mM Glutathione |
| 12 | 2.5% γ-Cyclodextrin + 15 mM Glutathione + 30 mM |
| | Histidine + 20 mM Cysteine |
| 13 | SBE-beta-Cyclodextrin + 15 mM Glutathione + 30 mM |
| | Histidine + 20 mM Cysteine |
| 14 | 4.5% SBE-beta-Cyclodextrin + 15 mM Glutathione + 30 mM |
| | Histidine + 150 mM Arginine |
| 15 | 2.5% γ-Cyclodextrin + 30 mM Histidine + 20 mM Cysteine |
| 16 | 4.5% SBE-beta-cyclodextrin + 30 mM Histidine + 20 mM Cysteine |
| 17 | 2.5% γ-Cyclodextrin + 30 mM Histidine + 150 mM Arginine |
| 18 | 4.5% SBE-beta-Cyclodextrin + 150 mM Arginine + 20 mM Cysteine |
| 19 | 4.5% SBE-beta-Cyclodextrin + 30 mM Histidine + 150 mM Arginine |
| 20 | 2.5% γ-Cyclodextrin + 150 mM Arginine + 20 mM |
| 21 | Cysteine 21 4.5% SBE-beta-Cyclodextrin + 150 mM Arginine |
| 22 | 2.5% γ-Cyclodextrin + 150 mM Arginine |

Consistent with previous excipient screening results described above, tIPV formulations containing one cyclodextrin (either 4.5% SBE-(β-Cyclodextrin or 2.5% γ-Cyclodextrin) in combination with 15 mM Glutathione or 30 mM Histidine had the lowest potency loss for each of the three IPV serotypes immediately after drying (Table 2.7 A). During 2-4 weeks storage at 4° C., for IPV1, the no excipient control (DPBS alone) lost about 60% potency, while formulations containing cyclodextrins (4.5% SBE-(β-Cyclodextrin or 2.5% γ-Cyclodextrin) in combination with 15 mM Glutathione achieved the lowest potency loss, which was less than 20%. For IPV2, the no excipient control lost about 30% potency, while all tested lead excipient combinations mitigated IPV2 potency loss during storage, and either cyclodextrin (4.5% SBE-βCyclodextrin or 2.5% γ-Cyclodextrin) in combination with 15 mM glutathione achieved the lowest potency loss. For IPV3, the no excipient control lost about lost about 50% potency, while either cyclodextrin (4.5% SBE-(β-Cyclodextrin or 2.5% γ-Cyclodextrin) in combination with 15 mM Glutathione achieved the lowest potency loss, which was less than 20%. Regarding the total potency loss of tIPV samples (after drying and 4 weeks storage at 4° C.), for IPV1, the no excipient control (DPBS alone) lost about 70% potency, while formulations containing cyclodextrins (4.5% SBE-(β-Cyclodextrin or 2.5% γ-Cyclodextrin) in combination with 15 mM Glutathione had the lowest total potency loss, which was less than 20%. Again, it was observed that formulations with 20 mM Cysteine caused over 20% potency loss in IPV1 immediately after drying and did not work well in preventing further potency loss during storage (40-55% total loss). For IPV2, the no excipient control lost about 30% potency but all tested lead excipient combinations worked well in preventing IPV2 potency loss after drying and storage. For IPV3, the no excipient control lost over 90% potency, while formulations containing either cyclodextrin (4.5% SBE-(3-Cyclodextrin or 2.5% γ-Cyclodextrin) in combination with 15 mM Glutathione had the lowest total potency loss (<25%). In addition, either formulations containing cyclodextrin (4.5% SBE-(β-Cyclodextrin or 2.5% γ-Cyclodextrin) in combination with 30 mM Histidine achieved less than 30% total potency loss for IPV3. Regarding the total potency loss after drying and after 4 weeks of accelerated storage at 25° C., potency loss for all three IPV serotypes in the no excipient control group was approximately 100%. In formulations containing cyclodextrin (either 4.5% SBE-(β-Cyclodextrin or 2.5% γ-Cyclodextrin) in combination with 15 mM Glutathione, total potency loss for IPV1, IPV2, and IPV3 was <25%, <35%, and <40%, respectively.

From the 4 week excipient combination screening study described above, we observed that the potency of IPV serotypes remained mostly consistent after the 2 week time point, suggesting that most of the potency loss during storage in the dried state occurred within the first 2 weeks post drying. We previously observed that for IPV3, most of the potency loss occurred within the first 24 hours post drying without excipient (FIG. 1.1). To better understand this phenomenon, potency loss for tIPV in the dried state was closely monitored within the first 2 weeks of storage post drying. The tIPV bulks were formulated in top candidate formulations (4.5% SBE-(β-Cyclodextrin or 2.5% γ-Cyclodextrin, each with 15 mM Glutathione, called F1 and F2, respectively) or without excipient (DPBS alone). D-antigen potency was tested immediately after drying, after 6 hrs, 1 day, 3 days, 7 days, and 14 days storage at 4° C. or at 25° C. Before analysis, the final pH of tIPV formulated in F1 and F2 were measured. As shown in Table 2.8, the pH of tIPV in original M199 media is 6.81, and the pH of tIPV formulated in F1 and F2 is 6.81 and 6.83, respectively.

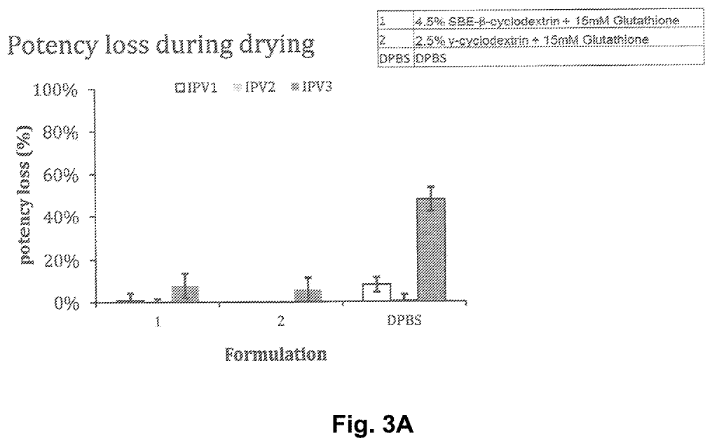

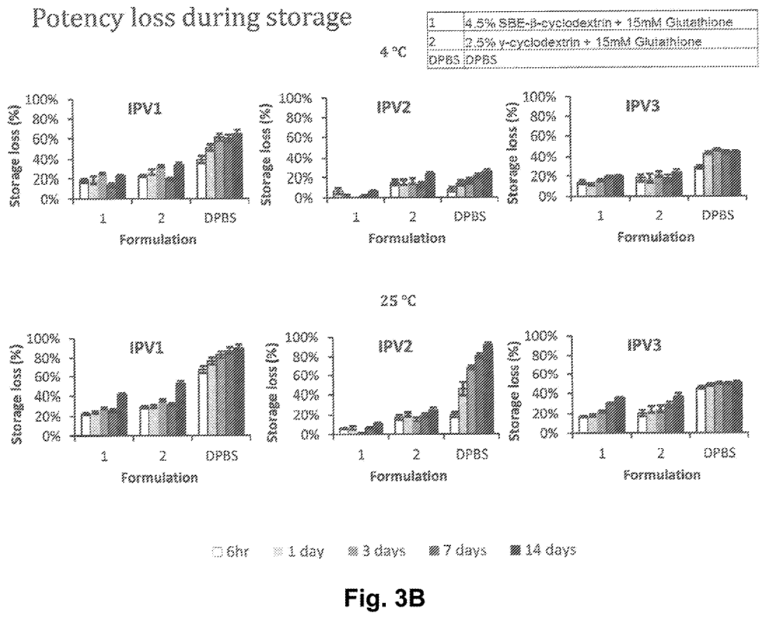

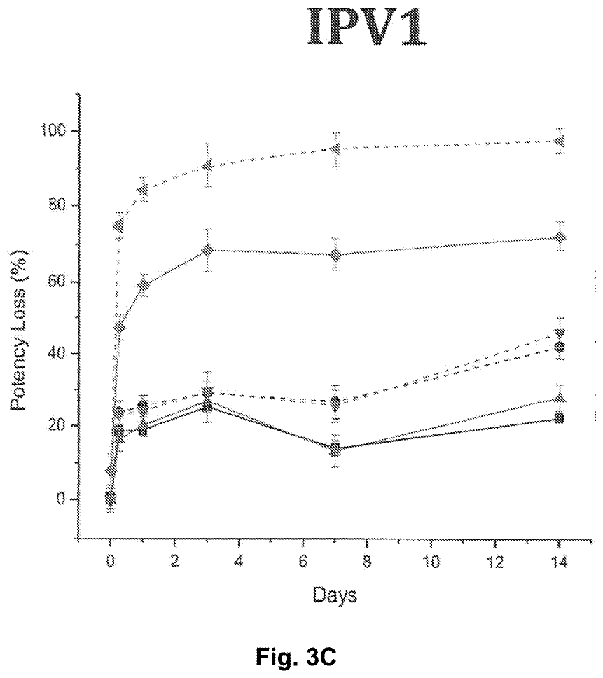

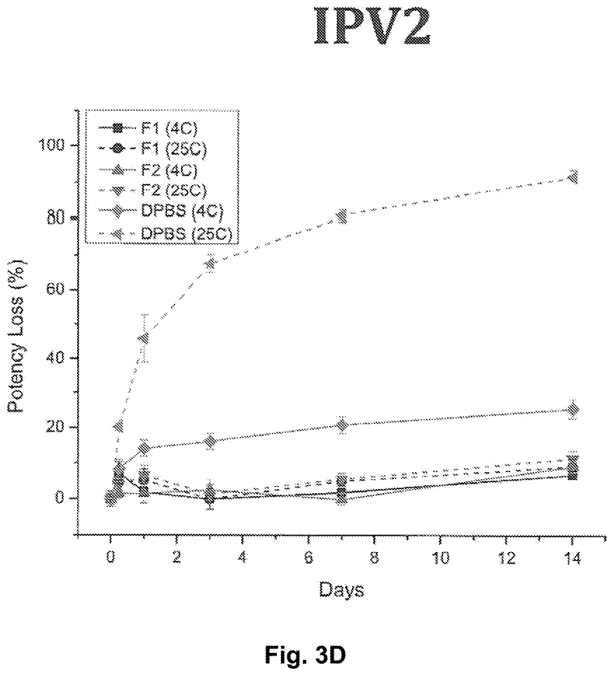

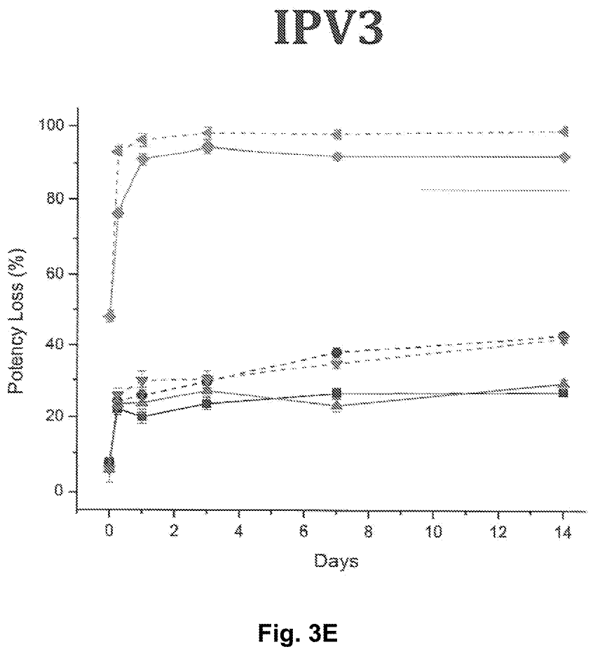

As shown in FIG. 3A, immediately after drying, IPV1, IPV2, and IPV3 in the DPBS control lost 10%, 0%, and 50% potency, respectively. In either candidate formulation, potency loss in IPV1, IPV2, and IPV3 was 0%, 0%, and 10%, respectively. During storage at 4° C., potency loss of tIPV in the DPBS control increased dramatically between time zero (immediately after drying) and the 6 hr time point for both IPV1 and IPV3 (potency loss in IPV2 was minimal between these time points) (FIG. 3B). Furthermore, potency loss continued at a lower rate between 6 hr and 3 days, and then leveled out between 3-14 days. Similar trends were observed in the two candidate tIPV formulations, albeit at much lower potency losses compared to the DPBS control. As observed previously, potency loss in the DPBS control was exacerbated at 25° C. but the potency loss in the candidate formulations were marginally higher during storage at 25° C. compared to 4° C. These results indicate the majority of potency losses in the three IPV serotypes in these top candidate formulations occurred within the first few days post-drying, particularly within the first few hours post-drying (FIG. 3C-3E).

The goal of Stage 2 was to develop top candidate formulations that stabilize tIPV vaccine during drying and storage. From the outset, two separate causes of D-antigen potency loss in the tIPV samples were expected. The first is the initial drying phase in which the tIPV was stressed as the bulk water is removed by evaporative drying (e.g., possible changes in ionic strength and pH). The second is the subsequent storage in the dried state when dried IPV may lose potency over time. Therefore, individual stabilizing excipients, identified from the initial excipient screening studies, were further studied and specified for their stabilizing ability with tIPV during drying and during subsequent storage. The combinations of the excipients for drying and the ones for storage provide protection for tIPV vaccine after drying and storage. Using this strategy, tIPV formulations which contain one cyclodextrin and glutathione were developed. Cyclodextrins were the best excipients identified for stabilizing IPV serotypes for drying, and glutathione was the most beneficial for improving tIPV stability during storage. The tIPV formulations containing combinations of one cyclodextrin and glutathione outperformed all single excipient formulations and other excipient combinations in terms of improving tIPV stability during drying and storage in the dried state.

tIPV formulations studied had the following composition: (1) 4.5% SBE (3-Cyclodextrin+15 mM Glutathione and (2) 2.5% γ-Cyclodextrin+15 mM Glutathione, both in M199/DPBS (a concentrated stock of excipients in DPBS, pH 7.2 is mixed with virus bulks in M199 medium, to obtain the targeted level of virus titer and excipient concentrations). Both of these two tIPV formulations maintained at least 90% D-antigen potency for all three IPV serotypes during drying (100%, 100%, and 90% potency for IPV1, 2, and 3 respectively), and at least 80% D-antigen potency in a dried state during 4 weeks storage at 4° C. (80%, 100%, and 80% potency for IPV1, 2, and 3 respectively); and at least 60% potency during 3 weeks of storage at 25° C. (70%, 100%, and 60% potency for IPV1, 2, and 3 respectively). Finally, a study to monitor potency loss during the first few weeks of storage after drying suggested that the loss rate is multi-phasic, and a majority of tIPV potency losses occur within the first few hours post drying. While long term stability studies are needed to better determine how these candidate tIPV formulations perform over the shelf-life of a potential commercial product, closely following potency loss over the first few days post drying could be used to potentially better understand the long-term stability profile of D-antigen potency loss in these candidate tIPV formulations.

During the preparation of samples for the four week stability study described above an additional plate was included in the tIPV samples incubated at 4° C., which covered approximately 50% of the excipient combinations tested in the study (Table 12).

TABLE 12
Excipient combinations tested with dried
tIPV after three months incubation at 4° C.
Excipient combinations tested with tIPV

2.5% γ-Cyclodextrin + 150 mM Arginine
2.5% γ-Cyclodextrin + 30 mM Histidine + 150 mM Arginine
2.5% γ-Cyclodextrin + 150 mM Arginine + 20 mM cysteine
2.5% γ-Cyclodextrin + 150 mM Arginine + 15 mM Glutathione
4.5% SBE-beta-cyclodextrin + 30 mM Histidine
4.5% SBE-beta-cyclodextrin + 30 mM Histidine
4.5% SBE-beta-cyclodextrin + 150 mM Arginine
4.5% SBE-beta-cyclodextrin + 20 mM Cysteine
4.5% SBE-beta-cyclodextrin + 15 mM Glutatione
4.5% SBE-beta-cyclodextrin + 30 mM Histidine + 20 mM Cysteine
4.5% SBE-beta-cyclodextrin + 150 mM Arginine + 20 mM Cysteine
4.5% SBE-beta-cyclodextrin + 150 mM Arginine + 15 mM Glutatione

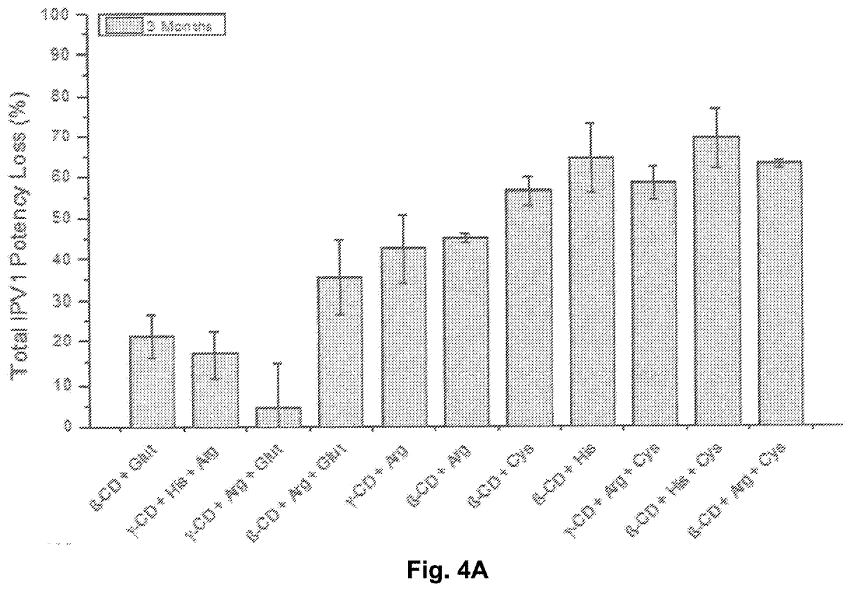

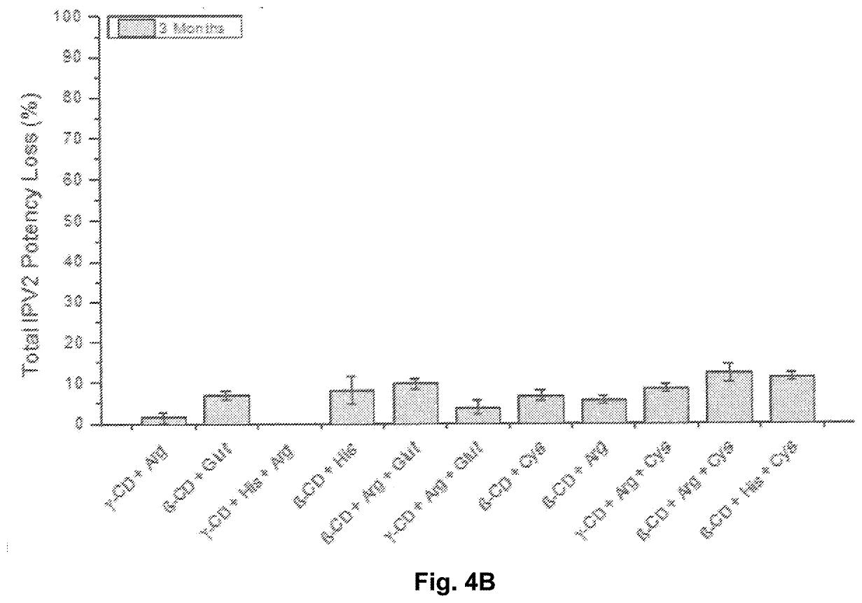

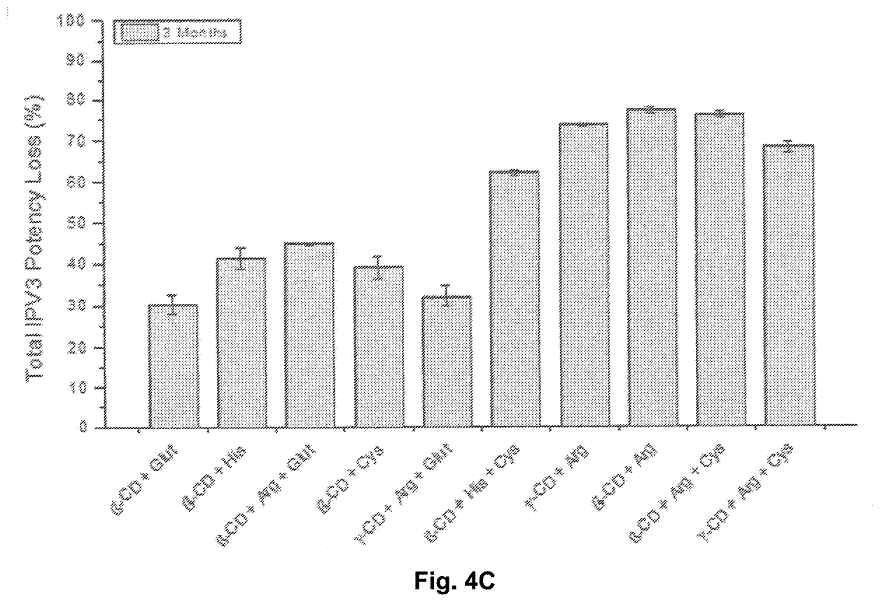

After three months of incubation at 4° C., the potency of each IPV serotype in each excipient condition was measured (FIGS. 4A-4C). The total potency loss of IPV1, IPV2, and IPV3 after three months in one of the two candidate formulations described above (4.5% SBE-(β-Cyclodextrin+15 mM Glutathione) was 21%, 7%, and 30%, respectively. These results support the observations that formulations stabilized tIPV and mitigated potency loss during drying and storage for up to 3 months at 4° C. (FIGS. 4A-4C).

### Example 6

Stability Testing of Various Influenza HA from Different Strains

Several strains of influenza were tested for stability in SBECD and arginine in various temperature and desiccant conditions. The results are tabulated below.

| | | | | | | | | HA | | | |
|---|---|---|---|---|---|---|---|---|---|---|---|
| | | | μg | TP | Exp. | Theo. | Theo | adj | | Drop in | Drop |
| | | | HA/mL | μg/mL | TP | HA | TP | for | Mean | pot to | in pot |
| Flu Strain | Excipient | Cond. | EIA | BCA | μg/mL | μg/mL | form. | TP | HA | target | to T₀ |
| B/Phuket | SBECD | 2-8° C. | 7.8 | 33.5 | | 15.3 | 41.2 | 9.6 | 9.8 | 35.8 | 0 |
| | | 1 des | | | | | | | | | |
| | | | 7.6 | 31.4 | 39.8 | | | 9.9 | | | |
| | | | 7.7 | 32.5 | | | | 9.7 | | | |
| | | | 7.7 | 31.4 | | | | 10.1 | | | |
| | | | 7.8 | 33.4 | | | | 9.7 | | | |
| | | 2-8° C. | | | | | | | | | |
| | | No des | | | | | | | | | |
| | | 25° C. | | | | | | | | | |
| | | 1 des | | | | | | | | | |

Table 13. B/Phuket in SBECD at time zero.

TABLE 14
B/Phuket in SBECD at T = 3 months

| | | | μg | TP | | Theo. | Theo | HA | | Drop in | Drop |
|---|---|---|---|---|---|---|---|---|---|---|---|
| | | | HA/mL | μg/mL | Exp. TP | HA | TP | adj | Mean | pot to | in pot |
| Flu Strain | Excipient | Cond. | EIA | BCA | μg/mL | μg/mL | form. | for TP | HA | target | to T₀ |
| B/Phuket | SBECD | 2-8° C. des | 5.8 | 34.2 | 40.2 | 15.3 | 41.2 | 6.0 | 3.0 | 80.6 | 69.8 |
| | | | 1.5 | 31.9 | | | | 1.7 | | | |
| | | | 2.7 | 35.7 | | | | 2.7 | | | |
| | | | 2.2 | 36.7 | | | | 2.1 | | | |
| | | | 2.3 | 33.4 | | | | 2.4 | | | |
| | | 2-8° C. No des | 2.1 | 30.7 | 40.2 | 15.3 | 41.2 | 2.4 | 2.8 | 81.9 | 71.8 |
| | | | 1.6 | 26.9 | | | | 2.1 | | | |
| | | | 4.4 | 43.5 | | | | 3.6 | | | |
| | | | 3.6 | 36.0 | | | | 3.5 | | | |
| | | | 1.5 | 23.2 | | | | 2.3 | | | |
| | | 25° C. des | 2.2 | 34.7 | 40.2 | 15.3 | 41.2 | 2.2 | 2.2 | 85.8 | 77.9 |
| | | | 1.0 | 25.9 | | | | 1.4 | | | |
| | | | 4.1 | 49.3 | | | | 2.9 | | | |
| | | | 1.8 | 33.0 | | | | 1.9 | | | |
| | | | 2.1 | 30.9 | | | | 2.4 | | | |

TABLE 15
A/Singapore in SBECD at T = 0 Month (time zero).

| | | | μg | TP | Exp. | Theo. | Theo | HA | | Drop in | Drop |
|---|---|---|---|---|---|---|---|---|---|---|---|
| | | | HA/mL | μg/mL | TP | HA | TP | adj | Mean | pot to | in pot |
| Flu Strain | Excipient | Cond. | EIA | BCA | μg/mL | μg/mL | form. | for TP | HA | target | to T₀ |
| A/Singapore | SBECD | 2-8° C. des | 7.7 | 29.2 | | 9.8 | 21.6 | 5.7 | 4.7 | 52.2 | 0 |
| | | | 5.1 | 42.8 | | | | 2.6 | | | |
| | | | 5.6 | 24.6 | | | | 4.9 | | | |
| | | | 6.2 | 27.1 | | | | 5.0 | | | |
| | | | 5.9 | 24.1 | | | | 5.3 | | | |
| | | 2-8° C. No des | | | | | | | | | |
| | | 2-8° C. 5 des | | | | | | | | | |
| | | 25° C. des | | | | | | | | | |
| | | 48° C. des | | | | | | | | | |

TABLE 16
A/Singapore in SBECD at T = 1 month

| | | | μg | TP | Exp. | Theo. | Theo | HA | | Drop in | Drop |
|---|---|---|---|---|---|---|---|---|---|---|---|
| | | | HA/mL | μg/mL | TP | HA | TP | adj | Mean | pot to | in pot |
| Flu Strain | Excipient | Cond. | EIA | BCA | μg/mL | μg/mL | form. | for TP | HA | target | to T₀ |
| A/Singapore | SBECD | 2-8° C. des | 6.5 | 32.2 | | 9.8 | 21.6 | 4.4 | 5.0 | 49.0 | −6.7 |
| | | | 6.7 | 24.4 | | | | 4.4 | | | |
| | | | 5.6 | 25.5 | | | | 6.0 | | | |
| | | | 6.5 | 25.1 | | | | 4.7 | | | |
| | | | 6.1 | 21.1 | | | | 5.6 | | | |
| | | 2-8° C. No des | | | | | | | | | |
| | | 2-8° C. 5 des | | | | | | | | | |
| | | 25° C. des | | | | | | | | | |
| | | 48° C. des | 6.0 | 22.6 | | 9.8 | 21.6 | 9.2 | 7.5 | 23.0 | −61.1 |
| | | | 6.2 | 29.1 | | | | 7.5 | | | |
| | | | 6.2 | 27.3 | | | | 8.0 | | | |
| | | | 4.9 | 37.0 | | | | 4.6 | | | |

TABLE 17
A/Singapore in SBECD at T = 3 months

| | | | μg | TP | Exp. | Theo. | Theo | HA | | Drop in | Drop |
|---|---|---|---|---|---|---|---|---|---|---|---|
| | | | HA/mL | μg/mL | TP | HA | TP | adj | Mean | pot to | in pot |
| Flu Strain | Excipient | Cond. | EIA | BCA | μg/mL | μg/mL | form. | for TP | HA | target | to T₀ |
| A/Singapore | SBECD | 2-8° C. des | 6.3 | 28.8 | | 9.8 | 21.6 | 7.8 | 6.3 | 35.8 | −34.4 |
| | | | 4.8 | 32.8 | | | | 5.1 | | | |
| | | | 6.3 | 28.1 | | | | 7.9 | | | |
| | | | 4.4 | 34.0 | | | | 4.6 | | | |
| | | | 5.1 | 29.6 | | | | 6.1 | | | |
| | | 2-8° C. No des | 5.6 | 36.1 | | 9.8 | 21.6 | 5.4 | 5.9 | 40.1 | −25.4 |
| | | | 5.7 | 30.7 | | | | 6.5 | | | |
| | | | 5.4 | 29.5 | | | | 6.4 | | | |
| | | | 4.5 | 34.2 | | | | 4.6 | | | |
| | | | 5.4 | 29.7 | | | | 6.4 | | | |
| | | 2-8° C. 5 des | 6.8 | 33.6 | | 9.8 | 21.6 | 7.1 | 6.4 | 34.9 | −36.2 |
| | | | 6.1 | 32.9 | | | | 6.5 | | | |
| | | | 6.0 | 32.5 | | | | 6.5 | | | |
| | | | 6.0 | 31.7 | | | | 6.7 | | | |
| | | | 4.8 | 32.6 | | | | 5.2 | | | |
| | | 25° C. des | 7.5 | 34.2 | | 9.8 | 21.6 | 7.7 | 6.8 | 30.3 | −45.9 |
| | | | 6.9 | 32.1 | | | | 7.5 | | | |
| | | | 6.1 | 31.8 | | | | 6.8 | | | |
| | | | 5.6 | 31.0 | | | | 6.3 | | | |
| | | | 5.0 | 30.6 | | | | 5.8 | | | |

TABLE 18
A/California in SEBCD at time zero

| | | | μg | TP | Exp. | Theo. | Theo | HA | | Drop in | Drop in |
|---|---|---|---|---|---|---|---|---|---|---|---|
| Flu | | | HA/mL | μg/mL | TP | HA | TP | adj for | Mean | pot to | pot to |
| Strain | Excipient | Cond. | EIA | BCA | μg/mL | μg/mL | form. | TP | HA | target | T₀ |
| A/Cal | SBECD | 2-8° C. des | 20.3 | 49.9 | 48.5 | 15.5 | 47.0 | 19.1 | 17.1 | −10.4 | 0 |
| | | | 16.6 | 52.9 | | | | 14.7 | | | |
| | | | 20.1 | 49.7 | | | | 19.0 | | | |
| | | | 19.3 | 64.0 | | | | 14.2 | | | |
| | | | 21.7 | 56.8 | | | | 18.5 | | | |
| | | 2-8° C. No des | | | | | | | | | |
| | | 25° C. des | | | | | | | | | |
| | | 48° C. des | | | | | | | | | |

TABLE 19
A/California in SEBCD at T = 1 month

| | | | μg | TP | | Theo. | Theo | HA | | Drop in | Drop in |
|---|---|---|---|---|---|---|---|---|---|---|---|
| Flu | | | HA/mL | μg/mL | Exp. TP | HA | TP | adj for | Mean | pot % | pot to |
| Strain | Excipient | Cond. | EIA | BCA | μg/mL | μg/mL | form. | TP | HA | to target | T₀ (%) |
| A/Cal | SBECD | 2-8° C. des | 18.5 | 44.2 | 47.2 | 15.5 | 47.0 | 19.7 | 17.6 | −13.4 | −2.7 |
| | | | 20.3 | 62.1 | | | | 15.4 | | | |
| | | | 21.8 | 50.2 | | | | 20.4 | | | |
| | | | 17.1 | 52.3 | | | | 15.4 | | | |
| | | | 19.4 | 53.3 | | | | 17.1 | | | |
| | | 2-8° C. No des | | | | | | | | | |
| | | 25° C. des | | | | | | | | | |
| | | 48° C. des | 12.4 | 37.4 | 47.2 | 15.5 | 47.0 | 15.6 | 14.9 | 3.7 | 12.7 |
| | | | 16.0 | 51.3 | | | | 14.6 | | | |
| | | | 15.5 | 52.5 | | | | 13.9 | | | |
| | | | 16.3 | 48.9 | | | | 15.7 | | | |

TABLE 20
A/California in SEBCD at T = 3 months

| | | | | | | | | | | | Drop |
|---|---|---|---|---|---|---|---|---|---|---|---|
| | | | μg | TP | | Theo. | Theo | HA | | Drop in | in pot |
| Flu | | | HA/mL | μg/mL | Exp. TP | HA | TP | adj | Mean | pot % to | to T₀ |
| Strain | Excipient | Cond. | EIA | BCA | μg/mL | μg/mL | form. | for TP | HA | target | (%) |
| A/Cal | SBECD | 2-8° C. des | 18.3 | 57.2 | 48.3 | 15.5 | 47.0 | 15.0 | 16.2 | −4.5 | 5.3 |
| | | | 19.7 | | | | | 14.5 | | | |
| | | | 15.9 | | | | | 14.4 | | | |
| | | | 34.3 | | | | | 18.6 | | | |
| | | | 38.6 | | | | | 18.4 | | | |
| | | 2-8° C. No des | 15.7 | | 48.3 | 15.5 | 47.0 | 17.1 | 14.1 | 8.8 | 17.3 |
| | | | 6.9 | | | | | 7.3 | | | |
| | | | 10.7 | | | | | 14.8 | | | |
| | | | 10.5 | | | | | 15.6 | | | |
| | | | 10.6 | | | | | 16.0 | | | |
| | | 25° C. des | 12.2 | | 48.3 | 15.5 | 47.0 | 16.2 | 12.7 | 18.1 | 25.8 |
| | | | 6.7 | | | | | 9.0 | | | |
| | | | 13.3 | | | | | 15.9 | | | |
| | | | 6.7 | | | | | 7.4 | | | |
| | | | 12.4 | | | | | 14.9 | | | |
| | | 48° C. des | | | | | | | | | |

TABLE 21
A/California in Arginine T = 0

| | | | μg | TP | Exp. TP | Theo. | Theo | | | Drop in | Drop |
|---|---|---|---|---|---|---|---|---|---|---|---|
| Flu | | | HA/mL | μg/mL | μg/mL | HA | TP | HA adj | Mean | pot to | in pot |
| Strain | Excipient | Cond. | EIA | BCA | | μg/mL | form. | for TP | HA | target | to T₀ |
| A/Cal | Arginine | 2-8° C. des | 18.8 | 48.7 | 52.8 | 17.0 | 51.6 | 19.9 | 18.9 | −11.1 | 0 |
| | | | 19.6 | 50.3 | | | | 20.1 | | | |
| | | | 18.7 | 47.6 | | | | 20.3 | | | |
| | | | 18.0 | 55.9 | | | | 16.6 | | | |
| | | | 16.0 | 46.7 | | | | 17.7 | | | |
| | | 25° C. des | | | | | | | | | |

TABLE 22
A/California in Arginine T = 3 months

| | | | | | | | | HA | | | |
|---|---|---|---|---|---|---|---|---|---|---|---|
| | | | μg | TP | | Theo. | Theo | adj | | Drop in | Drop |
| Flu | | | HA/mL | μg/mL | Exp. TP | HA | TP | for | Mean | pot to | in pot |
| Strain | Excipient | Cond. | EIA | BCA | μg/mL | μg/mL | form. | TP | HA | target | to T₀ |
| A/Cal | Arginine | 2-8° C. des | 18.0 | 44.7 | | 17.0 | 51.6 | 20.8 | 17.7 | −4.2 | 6.2 |
| | | | 13.9 | 62.9 | 50.9 | | | 11.4 | | | |
| | | | 17.7 | 48.8 | | | | 18.7 | | | |
| | | | 15.8 | 46.7 | | | | 17.5 | | | |
| | | | 18.9 | 48.3 | | | | 20.3 | | | |
| | | 25° C. des | 16.6 | 44.5 | 50.9 | 17.0 | 51.6 | 19.2 | 18.9 | −11.1 | 0.1 |
| | | | 20.0 | 48.4 | | | | 21.3 | | | |
| | | | 17.0 | 51.5 | | | | 17.0 | | | |
| | | | 18.1 | 54.2 | | | | 17.3 | | | |
| | | | 14.5 | 38.1 | | | | 19.6 | | | |

TABLE 23
A/South Australia in SBECD T = 0

| | | | μg | TP | | Theo. | Theo | HA adj | | Drop in | Drop |
|---|---|---|---|---|---|---|---|---|---|---|---|
| Flu | | | HA/mL | μg/mL | Exp. TP | HA | TP | for | Mean | pot to | in pot |
| Strain | Excipient | Cond. | EIA | BCA | μg/mL | μg/mL | form. | TP | HA | target | to T₀ |
| A/Cal | SBECD | 2-8° C. des | 21.4 | 44.1 | | 18.9 | | 17.1 | 20.4 | −7.9 | 0 |
| | | | 33.4 | 60.3 | 36.7 | | 35.1 | 19.5 | | | |
| | | | 28.6 | 43.4 | | | | 23.1 | | | |
| | | | 25.3 | 39.8 | | | | 22.3 | | | |
| | | | 23.8 | 41.7 | | | | 20.1 | | | |
| | | 2-8° C. No des | | | | | | | | | |
| | | 2-8° C. 5 des | | | | | | | | | |
| | | 25° C. des | | | | | | | | | |
| | | 48° C. des | | | | | | | | | |

TABLE 24
A/South Australia in SBECD T = 1 Month

| | | | μg | TP | Exp. | Theo. | Theo | HA adj | | Drop in | Drop |
|---|---|---|---|---|---|---|---|---|---|---|---|
| | | | HA/mL | μg/mL | TP | HA | TP | for | Mean | pot to | in pot |
| Flu Strain | Excipient | Cond. | EIA | BCA | μg/mL | μg/mL | form. | TP | HA | target | to T₀ |
| A/S | SBECD | 2-8° C. des | 22.4 | 32.4 | 31.9 | 18.9 | 35.1 | 24.3 | 21.6 | −14.4 | −6.0 |
| Australia | | | | | | | | | | | |
| | | | 19.7 | 36.7 | | | | 18.8 | | | |
| | | | 19.4 | 30.3 | | | | 22.5 | | | |
| | | | 17.9 | 29.0 | | | | 21.7 | | | |
| | | | 20.0 | 33.8 | | | | 20.8 | | | |
| | | 2-8° C. No des | | | | | | | | | |
| | | 2-8° C. 5 des | | | | | | | | | |
| | | 25° C. des | | | | | | | | | |
| | | 48° C. des | 25.7 | 40.4 | 31.9 | 18.9 | 35.1 | 22.4 | 21.9 | −15.9 | −7.4 |
| | | | 22.5 | 36.2 | | | | 21.9 | | | |
| | | | 21.1 | 33.5 | | | | 22.2 | | | |
| | | | 19.8 | 33.2 | | | | 21.0 | | | |
| | | | 22.2 | 35.3 | | | | 22.1 | | | |

TABLE 25
A/South Australia in SBECD T = 3 Months

| | | | μg | TP | Exp. | Theo. | Theo | | | Drop in | Drop |
|---|---|---|---|---|---|---|---|---|---|---|---|
| | | | HA/mL | μg/mL | TP | HA | TP | HA adj | Mean | pot to | in pot |
| Flu Strain | Excipient | Cond. | EIA | BCA | μg/mL | μg/mL | form. | for TP | HA | target | to To |
| A/S. | SBECD | 2-8° C. des | 15.9 | 32.0 | 28.8 | 18.9 | 35.1 | 17.4 | 17.9 | 5.4 | 12.3 |
| | | | 15.3 | 28.4 | | | | 19.0 | | | |
| | | | 14.9 | 27.6 | | | | 19.0 | | | |
| | | | 18.4 | 32.6 | | | | 19.8 | | | |
| | | | 24.0 | 59.2 | | | | 14.2 | | | |
| | | 2-8° C. No des | 21.1 | 49.2 | 28.8 | 18.9 | 35.1 | 15.1 | 16.0 | 15.5 | 21.6 |
| | | | 21.7 | 41.5 | | | | 18.4 | | | |
| | | | 14.5 | 43.7 | | | | 11.7 | | | |
| | | | 13.1 | 26.5 | | | | 17.4 | | | |
| | | | 15.0 | 30.3 | | | | 17.4 | | | |
| | | 2-8° C. 5 des | 15.5 | 32.5 | 28.8 | 18.9 | 35.1 | 16.7 | 17.5 | 7.4 | 14.2 |
| | | | 14.8 | 26.3 | | | | 19.8 | | | |
| | | | 14.6 | 33.8 | | | | 15.2 | | | |
| | | | 14.8 | 30.8 | | | | 16.9 | | | |
| | | | 14.2 | 26.7 | | | | 18.8 | | | |
| | | 25° C. des | 24.7 | 44.4 | 28.8 | 18.9 | 35.1 | 19.5 | 19.4 | −2.7 | 4.8 |
| | | | 16.8 | 32.5 | | | | 18.2 | | | |
| | | | 16.3 | 29.0 | | | | 19.8 | | | |
| | | | 15.7 | 26.4 | | | | 20.9 | | | |
| | | | 14.5 | 27.3 | | | | 18.7 | | | |
| | | 48° C. des | | | | | | | | | |

TABLE 26
Influenza in Arginine

| | HA CONCENTRATION | | | | | PROTEIN | | | |
|---|---|---|---|---|---|---|---|---|---|
| | (μg/mL) | | | | | CONTENT (μg/mL) | | | |
| TIME | A:Y1 | A:Y2 | A:Y3 | A:Y4 | A:Y5 | B:Y1 | B:Y2 | B:Y3 | B:Y4 |
| T0 | 19.3 | 18.0 | 19.0 | 18.7 | 19.1 | 42.46 | 41.41 | 41.05 | 40.91 |
| 7 D | 17.8 | 18.8 | 18.5 | 19.5 | 19.5 | 40.44 | 38.56 | 40.73 | 40.76 |
| 14 D | 18.1 | 19.2 | 17.0 | 18.3 | 16.6 | 43.68 | 48.75 | 42.27 | 46.57 |
| 28 D | 18.4 | 19.0 | 19.6 | 19.0 | 17.9 | 39.76 | 39.70 | 41.55 | 39.81 |

TABLE 27
Influenza in Arginine + SBECD

| | HA CONCENTRATION | | | | | PROTEIN | | | |
|---|---|---|---|---|---|---|---|---|---|
| | (μg/mL) | | | | | CONTENT (μg/mL) | | | |
| TIME | A:Y1 | A:Y2 | A:Y3 | A:Y4 | A:Y5 | B:Y1 | B:Y2 | B:Y3 | B: Y4 |
| T0 | 18.5 | 18.4 | 17.0 | 16.6 | 17.3 | 38.02 | 40.34 | 41.10 | 40.76 |
| 7 D | 19.3 | 20.4 | 19.3 | 20.8 | 18.7 | 43.69 | 45.96 | 45.45 | 47.60 |
| 14 D | 17.5 | 18.1 | 17.8 | 19.0 | 17.5 | 45.21 | 47.33 | 46.17 | 50.51 |
| 28 D | 16.6 | 17.5 | 18.5 | 18.0 | 19.8 | 37.62 | 38.92 | 38.39 | 41.23 |

TABLE 28
Influenza in polyvinylpyrrolidone

| | HA CONCENTRATION | | | | | PROTEIN | | | |
|---|---|---|---|---|---|---|---|---|---|
| | (μg/mL) | | | | | CONTENT (μg/mL) | | | |
| TIME | A:Y1 | A:Y2 | A:Y3 | A:Y4 | A:Y5 | B:Y1 | B:Y2 | B:Y3 | B:Y4 |
| T0 | 20.6 | 22.1 | 20.3 | 19.5 | 19.9 | 40.15 | 40.79 | 37.60 | 37.87 |
| 7 D | 19.2 | 18.0 | 18.7 | 18.4 | 17.3 | 36.52 | 36.42 | 39.27 | 36.16 |
| 14 D | 18.4 | 17.1 | 18.0 | 17.6 | 16.5 | 40.28 | 36.48 | 38.63 | 39.35 |
| 28 D | 18.7 | 20.3 | 18.1 | 21.3 | 19.8 | 34.99 | 37.00 | 35.28 | 38.44 |

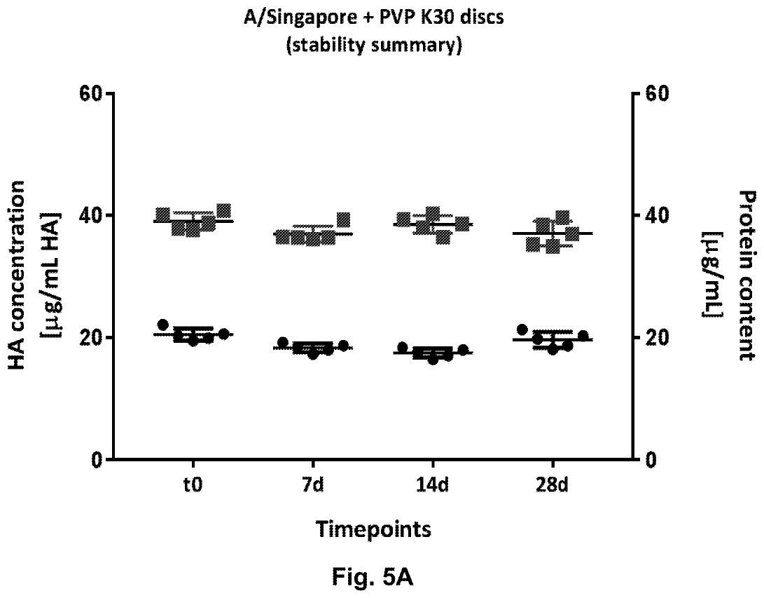

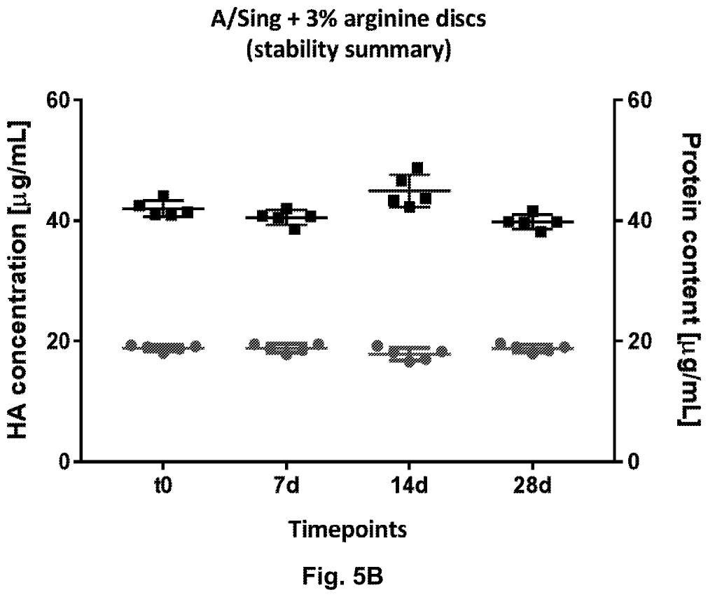

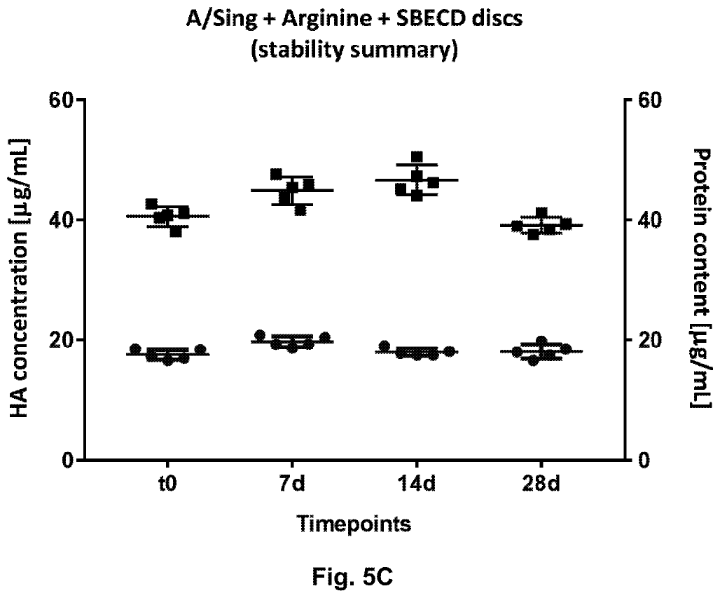

FIG. 5 shows plots of HA concentration and protein content versus various time points for A/Singapore in 1% polyvinylpyrrolidone, 3% arginine and 0.9% arginine and 0.3% SBECD on LCP discs at 2-8° C.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1. A high-density microprojection array comprising a base and a plurality of solid, non-porous and non-hollow microprojections, wherein the microprojections, wherein the distance between the microprojections is between 50 μm to 120 μm and wherein the microprojections are divided into at least a first section and a second section, each section comprising a plurality of microprojections, and wherein the microprojections in the first section are ink jet coated with a first substance, and wherein the microprojections in the second section are ink jet coated with a second substance.

2. The microprojection array of claim 1, wherein the first substance is a first multivalent vaccine and the second substance is a second multivalent vaccine.

3. The microprojection array of claim 1, wherein the first substance and the second substance are comprised of one or more vaccine antigens.

4. The microprojection array of claim 1, wherein the first substance is an antigen and the second substance is an adjuvant.

5. The microprojection array of claim 1, wherein the first substance is in a hydrophobic material and the second substance is a hydrophilic material.

6. The microprojection array of claim 1, wherein the first substance or the second substance contains a water soluble release substance.

7. The microprojection array of claim 1, wherein the first section has at least 100 microprojections.

8. The microprojection array of claim 7, wherein the second section has at least 100 microprojections.

9. The microprojection array of claim 1 wherein the first section has between 1000 to 10000 microprojections.

10. The microprojection array of claim 7, wherein the first section has between 1000 to 10000 microprojections.

11. The microprojection array of claim 10, wherein the microprojections are ink jet coated using a rotational spray coating and the microprojections are located on radial lines from a center point of rotation of the rotational spray coating.

12. The microprojection array of claim 10, wherein the microprojections are located on concentric circles.

13. The microprojection array of claim 10, wherein the microprojections are located on a continuous spiral.
